(19) United States
(12) Patent Application Publication
(10) Pub. No.: US 2024/0299566 A1
(43) Pub. Date: Sep. 12, 2024

(54) POLY-ARGININE DERIVATIVES FOR ENHANCING BRAIN-DERIVED GROWTH FACTOR TO MITIGATE NEUROLOGICAL DISORDERS

(71) Applicant: John Marshall, Barrington, RI (US)

(72) Inventor: John Marshall, Barrington, RI (US)

(21) Appl. No.: 18/260,440

(22) PCT Filed: Jan. 5, 2022
(86) PCT No.: PCT/US2022/011283

§ 371 Date: Jul. 5, 2023

Related U.S. Application Data
(60) Provisional application No. 63/134,059, filed on Jan. 5, 2021.

(51) Int. Cl. A61K 47/64; A61P 9/10; A61P 25/28; C07K 7/52

(52) U.S. Cl. CPC A61K 47/645; A61P 9/10; A61P 25/28; C07K 7/52

(57) ABSTRACT

Provided herein are compounds and compositions that are reversible agonists of the PDZ3 domain of PSD-95. Methods of their use in treating conditions of neural stress, inflammation, and viability are also provided.

### CROSS-REFERENCE TO RELATED APPLICATIONS

This application claims priority from Provisional Application No. 63/134,059, filed Jan. 5, 2021, the entire contents of which are hereby incorporated by reference.

### STATEMENT REGARDING FEDERALLY SPONSORED RESEARCH OR DEVELOPMENT

This invention was made with government support under grant number R41MH118747 awarded by the National Institute of Health. The government has certain rights in the invention.

### SEQUENCE LISTING

This application contains a Sequence Listing that has been submitted in ASCII format via EFS-Web and is hereby incorporated by reference in its entirety. The ASCII copy named 109549-713935_Sequence_Listing_ST25.txt was created on Jan. 4, 2021 and is 12 kilobytes in size.

### FIELD

The present disclosure is generally directed to reversible agonists of the PDZ3 domain of PSD-95 and their use in treating conditions of neural stress, inflammation, and viability.

### BACKGROUND

The PDZ domain is a common structural domain of 80-90 amino acids found in the signaling proteins of bacteria, yeast, plants, and animals. PDZ is an acronym combining the first letters of three proteins—post-synaptic density protein (PSD95), Drosophila disc large tumor suppressor (DlgA), and zonula occludens-1 protein (zo-1)—that were first discovered to share the domain. PDZ domains are also referred to as DHR (Dlg homologous region) or GLGF (glycine-leucine-glycine-phenylalanine) domains. These domains have been reported as helping to anchor transmembrane proteins to the cytoskeleton and hold together signaling complexes.

There are roughly 268 human PDZ domains in 151 distinct human proteins. One protein containing the PDZ domain is PSD-95, a tropomyosin receptor kinase B (TrkB)-associated synaptic scaffolding protein. Together with Shank3, SAP-90/PSD-95-associated protein (SAPAP1) and SynGAP, PSD-95 is highly enriched in synaptic regions, especially in the postsynaptic density (PSD), and is associated with neurological disorders, including major depressive disorder (MDD), autism spectrum disorder (Angelman syndrome) and schizophrenia. Together with synapse-associated protein 102 (SAP-102), postsynaptic density protein 93 (PSD-93), and SAP-97, PSD-95 is a core member of the MAGUK, with a common domain topology of three PDZ domains (PDZ1-3), an SH3 domain, and a GK domain.

### BRIEF SUMMARY

In accordance with an aspect of the disclosure, provided are compounds having a structure of Formula I, a salt thereof, and/or an isomer thereof:

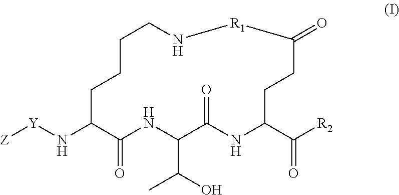

wherein R₁ comprises at least about one amino acid; R₂ is selected from the group consisting of isoleucine, leucine, alanine, phenylalanine, and valine; Y is a peptide chain comprising 1 to 10 amino acids; Z is hydrogen or a molecular transporter; and wherein the compound acts as a reversible agonist of the PDZ3 domain of PSD-95.

In some aspects, R₁ is alanine or β-alanine. In some cases, R₁ is β-alanine. In some cases, R₂ is valine or alanine. For example, R₂ may be valine.

In some aspects, Y comprises a peptide chain having a length of 3 to 9 amino acids. For example, Y may comprise any one of SEQ ID NOs: 1, 2 or 12-17. As another example, Y may comprise SEQ ID NO: 1 or 2. In various aspects, Y does not consist of SEQ ID NO: 3.

In various aspects, Z may comprise the molecular transporter. In some aspects, Z comprises a liposome, a steroid, a polyamine, a nanotube, a nanoparticle, a dendrimer, a cell penetrating peptide, a protein-transduction domain amino acid, a peptoid, (N-substituted glycine), an oligocarbamate, an arginine oligomer of about 6 to 20 units, a D-arginine oligomer, a spaced arginine oligomer, a N-arginine peptoid, an oligocarbamate transporter, or a tetrameric dendrimer. For example, in some aspects, Z may comprise a D-arginine oligomer, a spaced arginine oligomer, a N-arginine peptoid, an oligocarbamate transporter, a tetrameric dendrimer, a releasable-luciferin-transporter conjugate, a arginine oligomer of about 5 to 20 units, or a cell penetrating peptide.

In some aspects, Z may comprise the arginine oligomer of about 5 to 20 units. For example, in some aspects, the arginine oligomer can comprise about 7 to 15 arginine units. As an example, the arginine oligomer can comprise about 7 arginine units

In some aspects, Z may comprise the cell penetrating peptide. The cell penetrating peptide may comprise, in various aspects, any one of SEQ ID NOs: 4 to 11.

In any of the previous aspects, Y and Z may each comprise a cysteine and wherein Y and Z are connected by a disulfide bond. For example, in some aspects, Y and Z may together form a structure (Ia) or Structure (Ib):

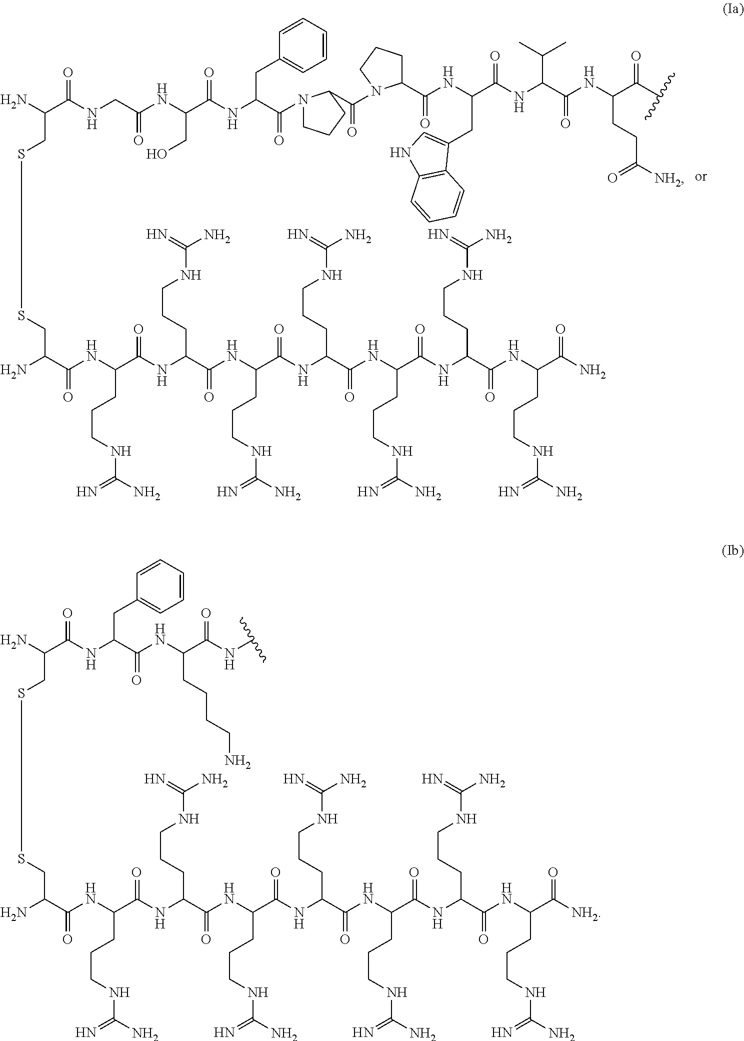 [SMILES: *C(=O)C(CCC(N)=O)NC(=O)C(NC(=O)C(Cc1c[nH]c2ccccc12)NC(=O)C1CCCN1C(=O)C1CCCN1C(=O)C(Cc1ccccc1)NC(=O)C(CO)NC(=O)CNC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O)C(C)C ; "or" ; *NC(=O)C(CCCCN)NC(=O)C(Cc1ccccc1)NC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O]

For example, the compound can have a structure in accordance with compounds of Formula (IV) or Formula (V).

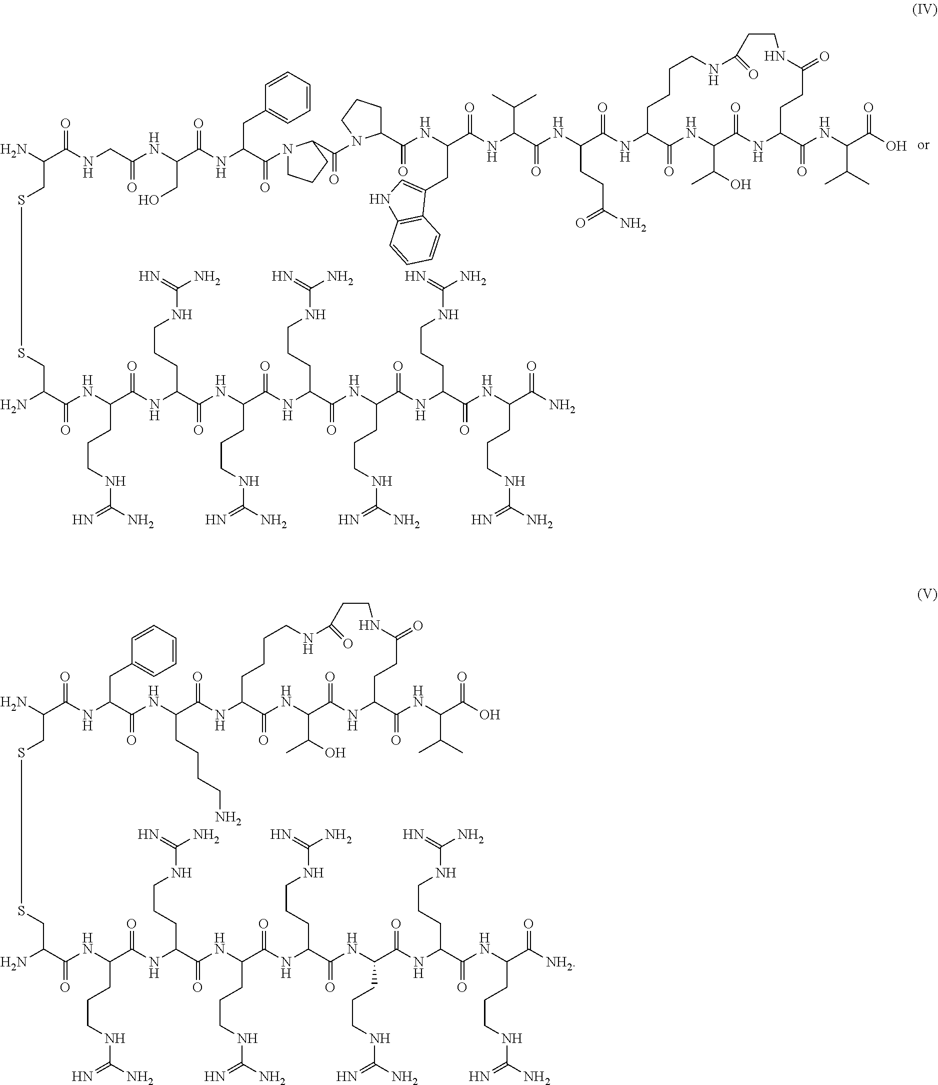

For example, the compound may be selected from:

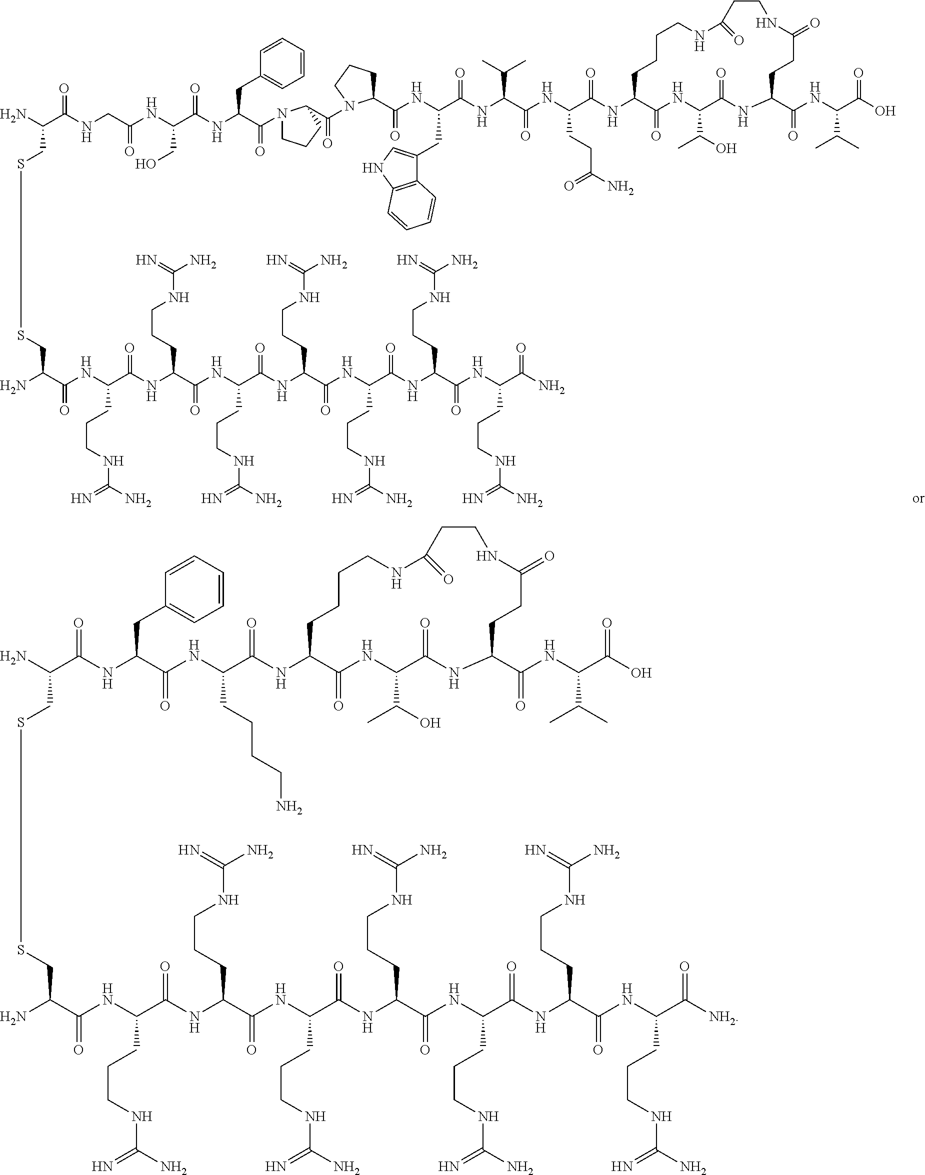

According to another aspect of the disclosure, provided are pharmaceutical compositions comprising a compound of Formula I, a salt thereof, or an isomer thereof, and a pharmaceutically acceptable excipient or carrier.

In some aspects, the composition may be prepared as a unit dosage form comprising from about 0.1 to about 100 mg of the compound of Formula I described herein, in the pharmaceutically acceptable excipient per unit dosage.

In accordance with another aspect of the disclosure, provided are methods for treating neural stress in a subject in need thereof, the methods comprising administering to the subject an effective amount of any compound of Formula I, a salt thereof or an isomer thereof.

In various aspects, the neuro-stress is selected from the group comprising depression, autism, schizophrenia, stroke, nerve crush, traumatic brain injury, epilepsy, pain or neurodegenerative disease. The neurodegenerative disease may be selected from the group consisting of retinal degeneration, Alzheimer's, and ALS.

In various aspects the effective amount of the compound may be from about 0.1 mg/kg to about 100 mg/kg according to the mass of the subject.

In various aspects, the route of administration may be intrathecal, parenteral, oral, buccal, sublingual or nasal. For example, the route of administration may be intrathecal.

### BRIEF DESCRIPTION OF THE DRAWINGS

FIG. 1A depicts western blots probed with phospho-specific antibodies against p-MTOR, p-Erk and p-S6 from SHSY5Y cells transfected with TrkB and stimulated with 30 ng/ml BDNF. Blots depict expression before (0′), or 15 or 60 minutes (15′ and 60′) after BDNF stimulation.

FIG. 1B is a bar graph quantifying relative expression of phosphorylated MTOR as determined by western blot in FIG. 1A.

FIG. 1C is a bar graph quantifying relative expression of phosphorylated ERK as determined by western blot in FIG. 1A.

FIG. 1D is a bar graph quantifying relative expression of phosphorylated S6 as determined by western blot in FIG. 1A.

FIG. 2A shows a plot of field EPSP slopes recorded in the CA1 region of the hippocampus in Compounds of Formula (IV) intraperitoneal (IP) injected Angelman syndrome (AS) mice at 2 mg/kg. LTP was observed.

FIG. 2B shows a plot of field EPSP slopes recorded in the CA1 region of the hippocampus in Compounds of Formula (IV) intraperitoneal (IP) injected AS mice at 1 mg/kg. LTP was observed.

FIG. 2C shows a plot of field EPSP slopes recorded in the CA1 region of the hippocampus in Compounds of Formula (IV) intraperitoneal (IP) injected AS mice at 0.5 mg/kg. LTP was observed.

FIG. 2D shows a plot of field EPSP slopes recorded in the CA1 region of the hippocampus in CN2097 intraperitoneal (IP) injected AS mice at 1 mg/kg. No LTP was observed.

FIG. 3A is a scatter plot showing immobility time of control mice or depressed mice during a Forced Swim Test, 7 days after injection of vehicle or Compounds of Formula (IV), where *P<0.001 vs. control mice.

FIG. 3B is a scatter plot showing immobility time of control mice or depressed mice during a Tail Suspension Test, 7 days after injection of vehicle or Compounds of Formula (IV), *P<0.001 vs. control mice.

FIG. 4A shows a bar graph depicting IL-10 expression after acute injection of Compounds of Formula (IV) in a mouse model of depression.

FIG. 4B shows a bar graph depicting IL-6 expression after acute injection of Compounds of Formula (IV) in a mouse model of depression.

FIG. 4C shows images depicting microglia in control mice, or in a mouse model of depression either alone or after treatment with an acute injection of Compounds of Formula (IV).

### DETAILED DESCRIPTION

The terms “polypeptide,” “peptide” and “protein” are used interchangeably herein to refer to a polymer of amino acid residues. The terms are to be broadly construed also comprising amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma.-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid such as β-alanine.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Aspects of the disclosure are directed to compounds of Formula (I). As used herein, compounds of Formula (I) may refer salts thereof, isomers thereof, prodrugs thereof, and/or the like. Although not expressly stated, embodiments of compounds of Formula (I) may exclude one or more of the salts thereof, the isomers thereof, and/or prodrugs thereof in some instances. The compounds of Formula (I) may be reversible agonists of the PDZ domain of PSD-95. Accordingly, provided herein is a compound of Formula (I):

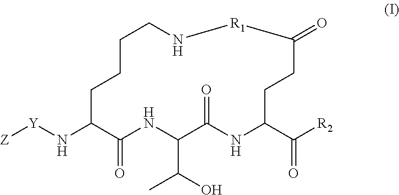

wherein R₁ comprises at least about one amino acid; R₂ is selected from the group consisting of isoleucine, leucine, alanine, phenylalanine, and valine; Y is a peptide chain comprising 1 to 10 amino acids; Z is hydrogen or a molecular transporter; and wherein the compound acts as a reversible agonist of the PDZ3 domain of PSD-95. In some cases, when R₁ is β-alanine, R₂ is valine, and Z comprises a poly-arginine chain and a disulfide bond connected to Y, Y does not comprise SEQ ID NO: 3 (CKNYK).

The compounds and compositions comprising the structure of Formula (I) have advantageously been shown to reversibly bind the PDZ3 domain of PSD-95. As such they can be useful agents to modulate PSD-95 signaling-particularly as it relates to BDNF signaling, as described below. The compounds also show a structural core (Y) with amino acids that advantageously bind PDZ3 domains containing this C-terminal helix extension (aa 306-S412, referred to as “PDZ3C”) with a 10-15-fold higher than related compounds without the core.

The compounds of Formula (I) typically include a cyclic moiety between Y and R₂. This cyclic moiety may bind to all three PDZ domains in PSD-95. Referring to Formula (I), above, the cyclic moiety comprises two amino acids joined to R₁. Preferably, the two amino acids are joined to R₁ by a covalent linkage. As shown in Formula (I) the two amino acids joined to R₁ are glutamic acid and lysine. R₁ is typically at least one amino acid. In some cases, R₁ comprises from a single amino acid to at least 6 amino acids, though particular note is made of R₁ comprising a single amino acid, two amino acids, or three amino acids. For example, R₁ may be selected from alanine, glycine, isoleucine, leucine, methionine, proline, valine, tryptophan, and a combination thereof. In some cases, R₁ may be selected from alanine, glycine, isoleucine, leucine, proline, valine, and a combination thereof. In further cases, R₁ may be selected from alanine, glycine, methionine, tryptophan, and a combination thereof. R₁ preferably comprises alanine. In at least one embodiment, R₁ comprises is beta-alanine (β-alanine). Further description of suitable cyclic moieties for compounds of Formula (I) are provided in U.S. Pat. No. 10,046,024, which is incorporated herein by reference in its entirety for all purposes.

R₂ may be any amino acid. For example, R₂ may be selected from isoleucine, leucine, alanine, phenylalanine, valine, and a combination thereof. In some cases, R₂ may be selected from valine, alanine, or a combination thereof. In at least one embodiment, R₂ is valine.

Y is typically a moiety that comprises a linker amino acid region. Preferably, Y includes amino acids that bind to a C-terminal a-helix amino acids beyond the conventional PDZ3 domain structural core. The linker region(s) of Y preferably bind PDZ3 domains containing C-terminal a-helix extension (aa 306-S412, referred to as “PDZ3C”) with a multiple of 10 to 15-fold greater affinity than the PDZ3 domain alone.

Y can comprise a peptide chain of 1 to 10 amino acid(s). For example, Y can comprise 3 to 9 amino acids, 3 to 8 amino acids, 3 to 7 amino acids, 3 to 6 amino acids, 3 to 5 amino acids; 4 to 9 amino acids, 4 to 8 amino acids, 4 to 7 amino acids, 4 to 6 amino acids; 5 to 9 amino acids, 5 to 8 amino acids, 5 to 7 amino acids; 6 to 9 amino acids, 6 to 8 amino acids; or 7 to 9 amino acids. In some embodiments, Y comprises 3 amino acids, 4 amino acids, 5 amino acids, 6 amino acids, 7 amino acids, 8 amino acids, or 9 amino acids.

In at least one embodiment, Y is 3 amino acids. Y may comprise 3 to 9 amino acids selected from cysteine (Cys), glycine (Gly), serine (Ser), phenylalanine (Phe), proline (Pro), threonine (Trp), valine (Val), glutamine (Gln), and a combination thereof. In some instances, Y comprises Cys, Gly, Ser, Phe, Pro, Trp, Val, and Gln, e.g., arranged in any order. For instance, Y can comprise SEQ ID NO: 1 (Cys-Gly-Ser-Phe-Pro-Pro-Trp-Val-Gln). Alternatively, Y can comprise Cys-Gly-Ser-Phe-Pro-Pro-Trp-Val-Lys (SEQ ID NO: 12), Cys-Gly-Ser-Phe-Pro-Pro-Trp-Val-Lys-Lys (SEQ ID NO: 13), Cys-Gly-Ser-Phe-Pro-Pro-Trp-Val-Lys-Gln (SEQ ID NO: 14) Cys-Gly-Ser-Phe-Pro-Pro-Trp-Val-Gln-Lys (SEQ ID NO: 15) Cys-Gly-Ser-Phe-Pro-Pro-Trp-Val-Gln-Gln (SEQ ID NO: 16), Alternatively, Y may comprise Cys, Tyr, and Lys, e.g., arranged in any order. In at least one embodiment, Y can comprise SEQ ID NO: 2 (Cys-Tyr-Lys). Preferably, Y does not comprise SEQ ID NO: 3 (CKNYK). In one embodiment, when R₁ is β-alanine, R₂ is valine, and Z comprises a poly-arginine chain and a disulfide bond connected to Y, Y does not comprise SEQ ID NO: 3 (CKNYK). In at least one embodiment, Y comprises at least one D amino acid. For instance, Y can comprise dCys-Gly-Ser-Phe-dPro-Pro-Trp-Val-Gln (SEQ ID NO: 17), where dCys and dPro are D amino acids.

Z typically comprises a hydrogen or a molecular transporter. Z may be a molecular transporter that facilitates entry of the compounds of Formula (I) into a human cell. In various instances, Z comprises a liposome, a steroid, a polyamine, a nanotube, a nanoparticle, a dendrimer, a cell-penetrating peptide, a protein-transduction domain amino acid, a peptoid, (N-substituted glycine), an oligocarbamate, an arginine oligomer of about 5 to 20 units, a D-arginine oligomer, a spaced arginine oligomer, an N-arginine peptoid, an oligocarbamate transporter, or a tetrameric dendrimer. For example, the Z may comprise a D-arginine oligomer, a spaced arginine oligomer, a N-arginine peptoid, a oligocarbamate transporter, a tetrameric dendrimer, a releasable-luciferin-transporter conjugate, an arginine oligomer of about 6 to 20 units, or a cell penetrating peptide. In some cases, Z can comprise the arginine oligomer. The arginine oligomer can comprise about 7 to 15 arginine units. For example, the oligomer can comprise about 7 arginine units.

Additionally, or alternatively, Z can comprise a cell-penetrating peptide. Cell-penetrating peptides are well understood in the art (see e.g., Stewart et al., “Cell-penetrating peptides as deliver vehicles for biology and medicine,” Org. Biomol. Chem., 6:2242-2255 (2008); The Handbook of Cell-Penetrating Peptides, Second Edition, Ülo Langel, Ed, CRC (2006); and Cell-Penetrating Peptides: Processes and Applications, Ülo Langel, Ed, CRC (2002), which are each incorporated by reference herein in their entirety for all purposes). Z may comprise any of cell-penetrating peptide listed in Table 1, below. In some cases, Z may comprise an amino acid sequence of a cell-penetrating peptide selected from SEQ ID NOs: 4 to 11.

TABLE 1

| Cell-penetrating | | SEQ |
|---|---|---|
| peptide | Amino acid sequence | ID NO: |
| Polyarginines | RRRRRRRRR (R₉) | 4 |
| Tat₄₉₋₅₇ | RKKRRQRRR | 5 |
| Penetratin | RQIKIWFQNRRMKWKK | 6 |
| (Antennapedia) | | |
| Pep-1 | KETWWETWWTEWSQPKKKRKV | 7 |
| Transportan | GWTLNSAGYLLGKINLKALAALAKKIL | 8 |
| Nuclear | VQRKRQKLMP | 9 |
| localization | SKKKKIKV | 10 |
| sequences | GRKRKKRT | 11 |

Y and Z are typically connected via a bridge element. The bridge element generally comprises an effective bond between the terminal ends of Y and Z. In at least one embodiment, Z may, e.g., in addition to the above, further comprise a cysteine residue. In some aspects, the cysteine residue is a D-amino acid. This enables, for example, connecting Z to a sulfur in Y (such as, for example, one present in a cysteine residue) with a disulfide bond. Without being bound by any theory, this disulfide bond may provide additional benefits to the compound. For example, it may enable the PSD-95 binding moieties to separate from the poly-arginine moiety, allowing the resulting polyarginine-cysteine complex (Z) to facilitate blood-brain barrier permeation and provide additional therapeutic benefits by affecting the metabolism of mitochondria.

In some embodiments, Y and Z can together form structure (Ia) or (Ib), as shown below.

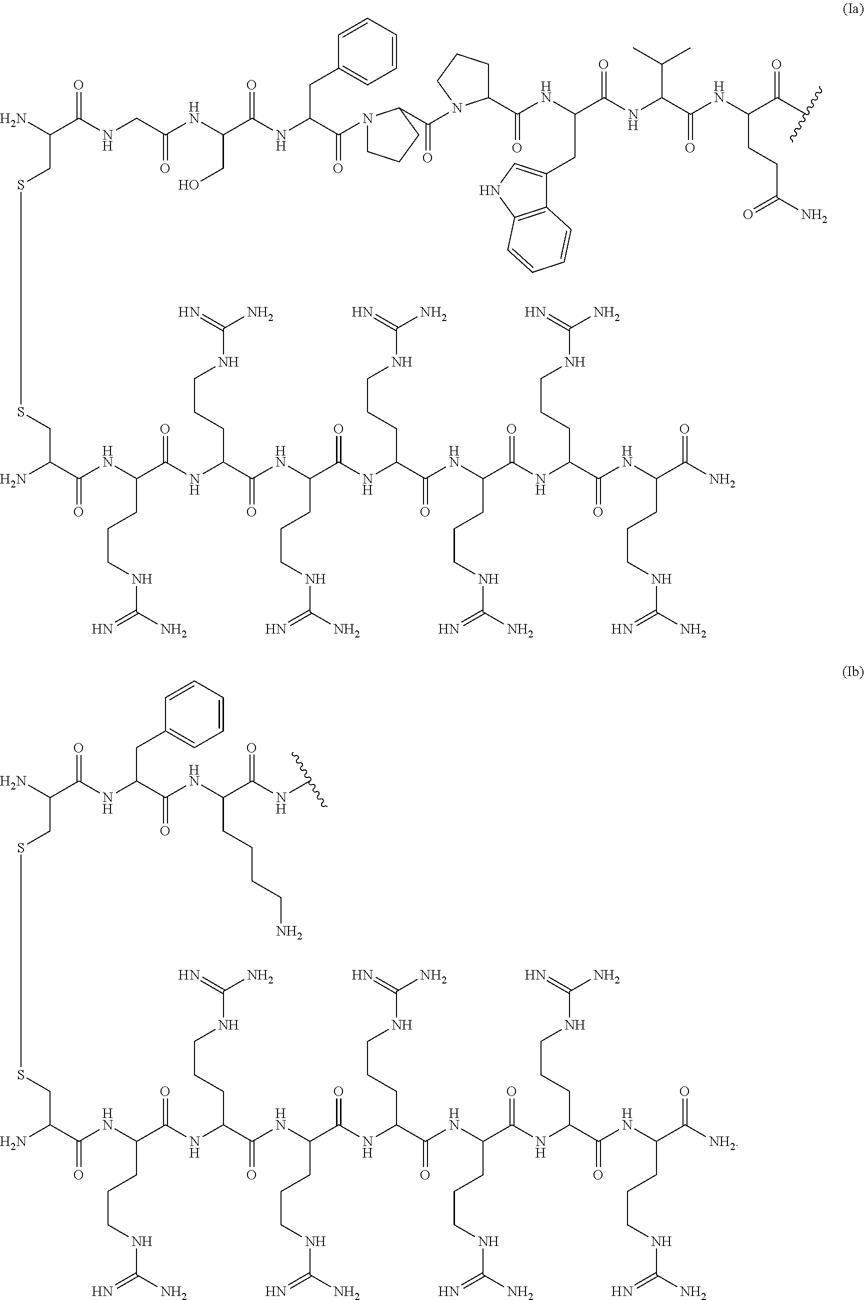 [SMILES: *C(=O)C(CCC(N)=O)NC(=O)C(NC(=O)C(Cc1c[nH]c2ccccc12)NC(=O)C1CCCN1C(=O)C1CCCN1C(=O)C(Cc1ccccc1)NC(=O)C(CO)NC(=O)CNC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O)C(C)C ; *NC(=O)C(CCCCN)NC(=O)C(Cc1ccccc1)NC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O]

The compounds of Formula (I) may have a structure according to Formula (II), provided below, where R₁, R₂, and Z are groups as disclosed herein.

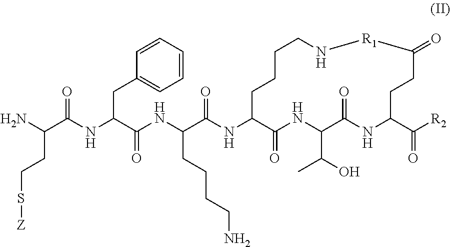

In some embodiments, the compounds of Formula (II) have a structure according to Formula (IIa), as shown below, where R₁, and R₂, are groups as disclosed herein.

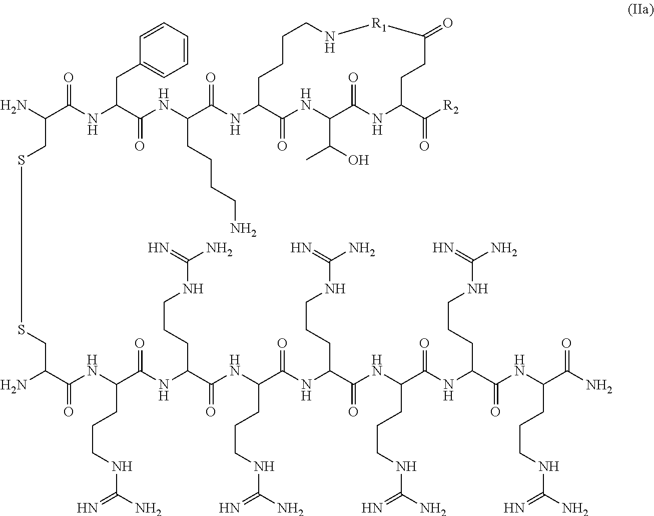

In other embodiments, the compounds of Formula (II) have a structure according to Formula (IIb), as shown below, where R₁, and Z are groups as disclosed herein.

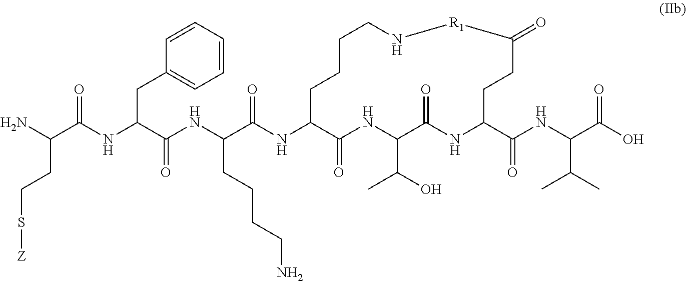

In other embodiments, the compounds of Formula (II) have a structure according to Formula (IIc), as shown below, where R₂ and Z are groups as disclosed herein.

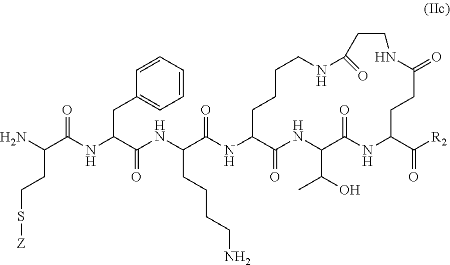

The compounds of Formula (I) may have a structure according to Formula (III), provided below, where R₁, R₂, and Z are groups as disclosed herein.

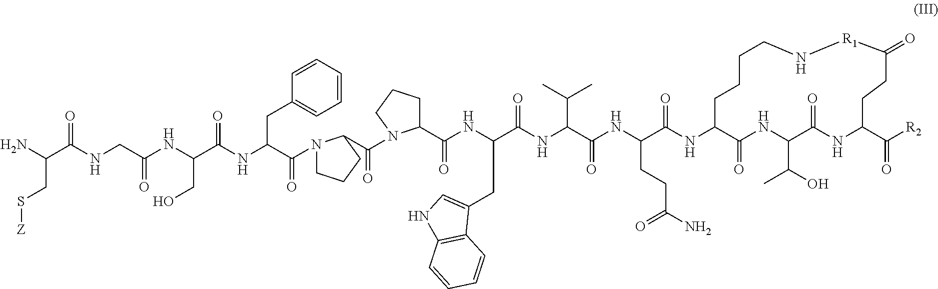

The compounds of Formula (III) may have a structure according to Formula (IIIa) provided below, where R₁, and R₂, are groups as disclosed herein.

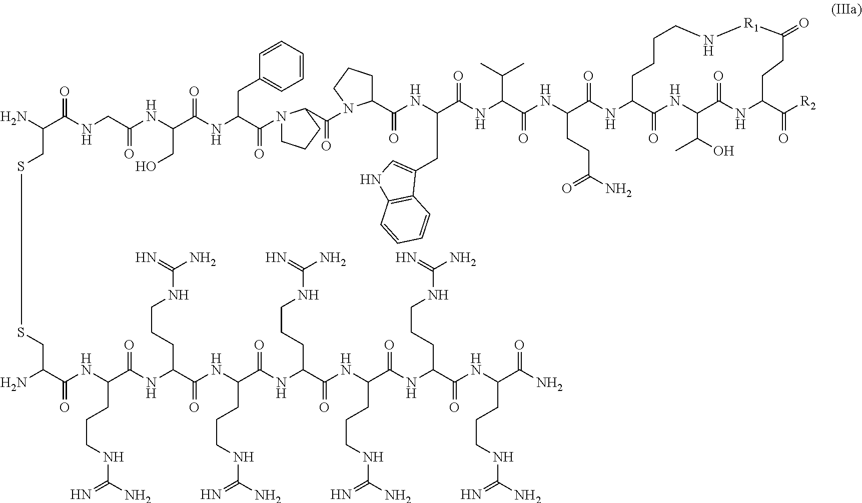

The compounds of Formula (III) may have a structure according to Formula (IIIb), provided below, where Z and R₂ are groups as disclosed herein.

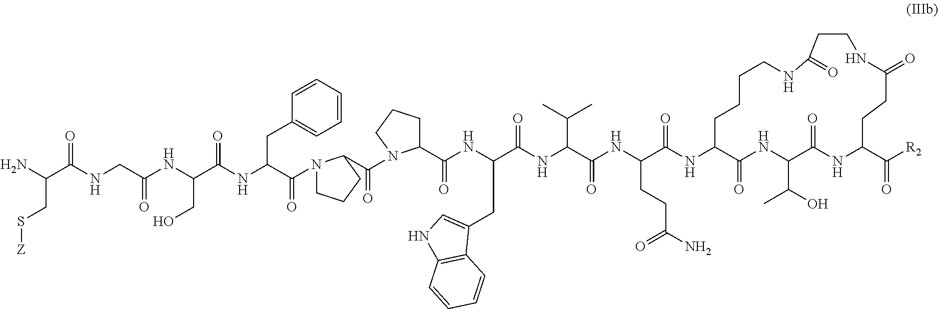

The compounds of Formula (III) may have a structure according to Formula (IIIc), provided below, where Z and R₁ are groups as disclosed herein.

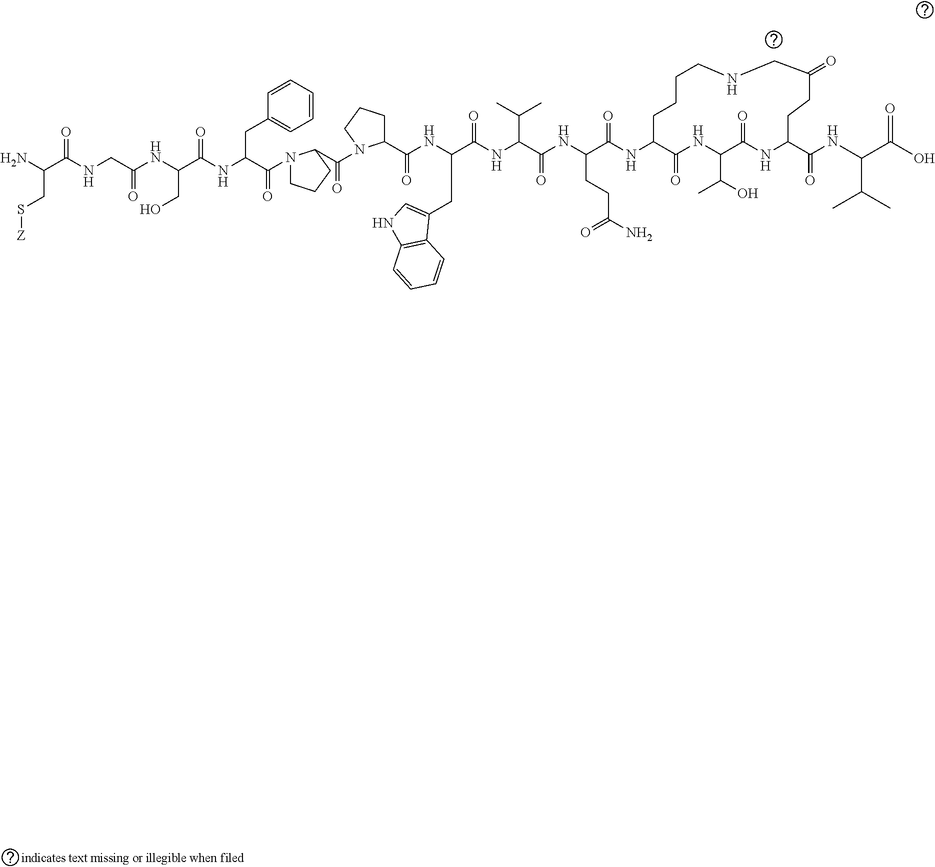

In some instances, the compounds of Formula (I) can have a structure selected from Formula (IV) or Formula (V), which are shown below.

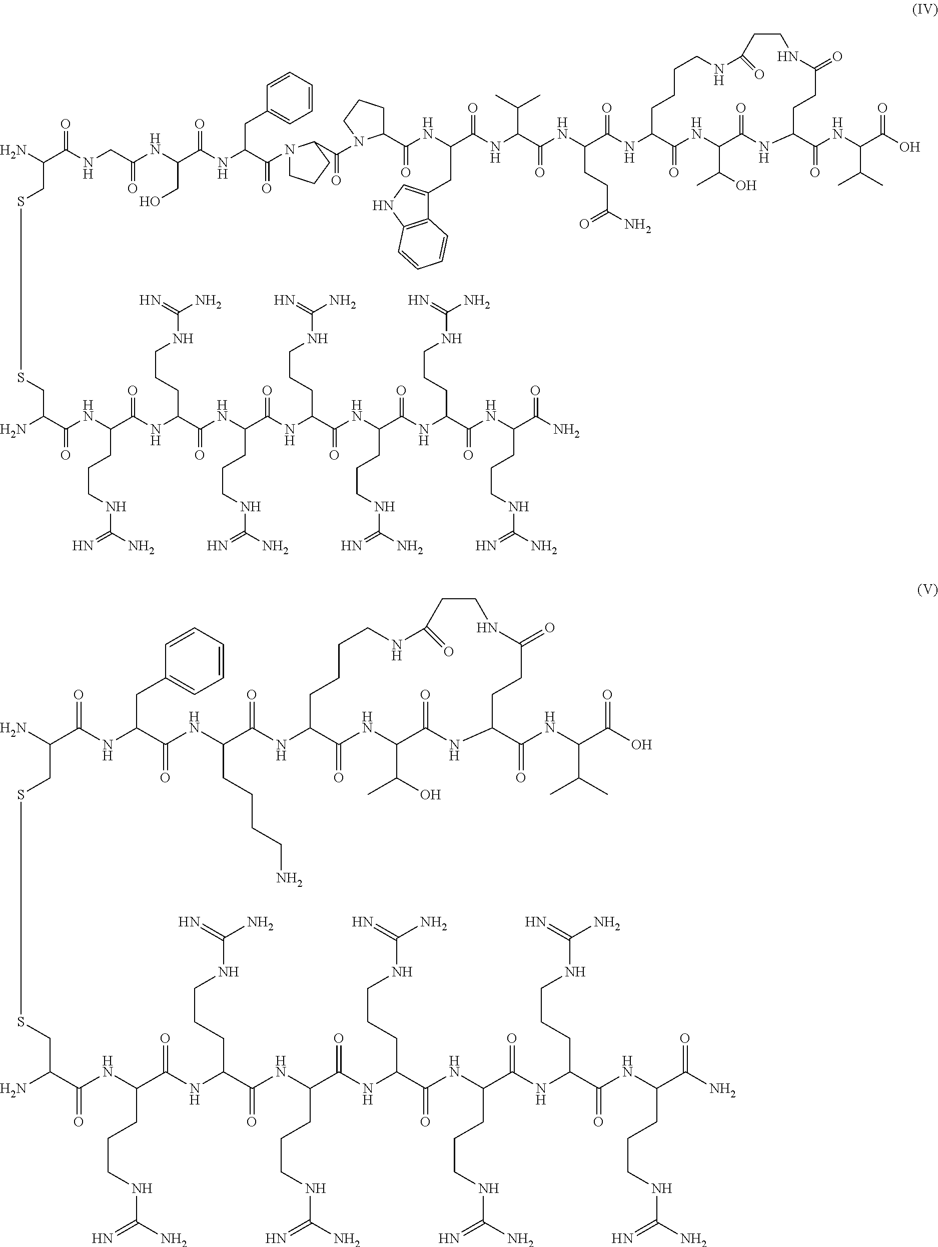

In some instances, the compounds of Formula (I) can have a structure selected from.

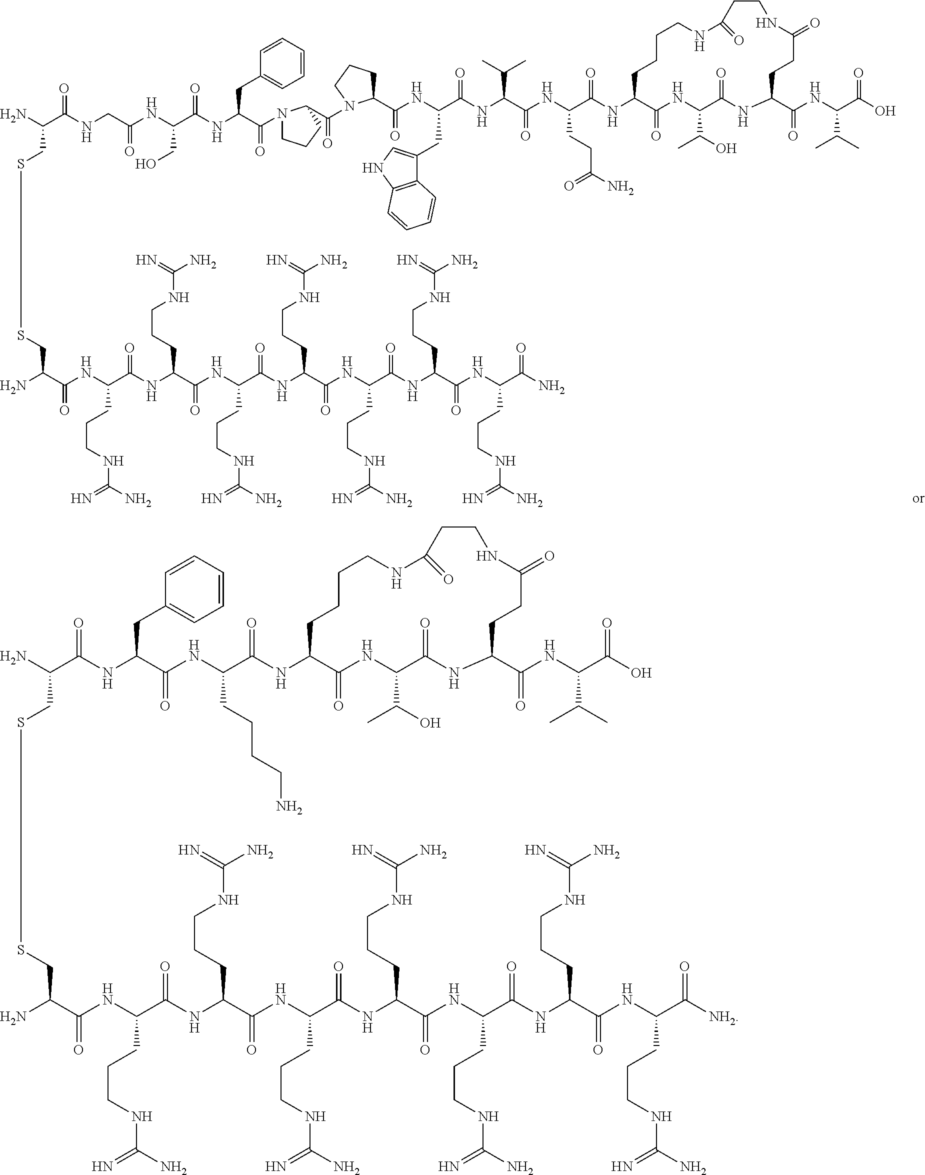

Various methods for producing cyclic peptides have been described. Examples of chemical reaction protocols for producing circularized peptides are described in U.S. Pat. Nos. 4,033,940 and 4,102,877, which are both incorporated herein by reference in their entirety for all purposes. In other techniques, biological and chemical methods are combined to produce cyclic peptides. Some methods involve first expressing linear precursors of cyclic peptides in cells (e.g., bacteria) to produce linear precursors of cyclic peptides and then adding of an exogenous agent such as a protease or a nucleophilic reagent to chemically convert these linear precursors into cyclic peptides. Examples of such methods are described in Camerero, J. A., and Muir, T. W., J. Am. Chem. Society. 121:5597 (1999); Wu, H. et al, Proc. Natl. Acad. Sci. USA, 95:9226 (1998); and Martin Linhult et al., “Evaluation of different linker regions for multimerization and coupling chemistry for immobilization of a proteinaceous affinity ligand,” Protein Engineering, vol. 16 no. 12 pp. 1147-1152, Oxford University Press (2003), which are incorporated by reference herein in their entirety for all purposes.

### Pharmaceutical Compositions

As noted, various aspects of the present disclosure relate to pharmaceutical compositions comprising an amount of a compound of Formula (I), a salt thereof, and/or a prodrug thereof and a pharmaceutically acceptable carrier or excipient. The amount of Formula (I), a salt thereof, and/or a prodrug thereof present in the pharmaceutical compositions may be a therapeutically effective amount. As used herein “therapeutically effective amount” or “therapeutically effective dosage” refers to an amount that is effective to achieve a desired therapeutic result.

Generally, the compositions or formulations described herein can be prepared as unit doses comprising about 0.1 to about 100 mg of the compound per unit dosage in a suitable carrier or excipient. In some cases, the amount of compound of Formula (I) present in pharmaceutical composition is about 0.1 to about 100 mg, about 1 to about 100 mg, about 5 to about 100 mg, about 10 to about 100 mg, about 15 to about 100 mg, about 20 to about 100 mg, about 25 to about 100 mg, about 30 to about 100 mg, about 35 to about 100 mg, about 40 to about 100 mg, about 45 to about 100 mg, about 50 to about 100 mg, about 55 to about 100 mg, about 60 to about 100 mg, about 65 to about 100 mg, about 70 to about 100 mg, about 75 to about 100 mg, about 80 to about 100 mg, about 85 to about 100 mg, about 90 to about 100 mg; about 0.1 to about 90 mg, about 1 to about 90 mg, about 5 to about 90 mg, about 10 to about 90 mg, about 15 to about 90 mg, about 20 to about 90 mg, about 25 to about 90 mg, about 30 to about 90 mg, about 35 to about 90 mg, about 40 to about 90 mg, about 45 to about 90 mg, about 50 to about 90 mg, about 55 to about 90 mg, about 60 to about 90 mg, about 65 to about 90 mg, about 70 to about 90 mg, about 75 to about 90 mg, about 80 to about 90 mg; about 0.1 to about 80 mg, about 1 to about 80 mg, about 5 to about 80 mg, about 10 to about 80 mg, about 15 to about 80 mg, about 20 to about 80 mg, about 25 to about 80 mg, about 30 to about 80 mg, about 35 to about 80 mg, about 40 to about 80 mg, about 45 to about 80 mg, about 50 to about 80 mg, about 55 to about 80 mg, about 60 to about 80 mg, about 65 to about 80 mg, about 70 to about 80 mg; about 0.1 to about 70 mg, about 1 to about 70 mg, about 5 to about 70 mg, about 10 to about 70 mg, about 15 to about 70 mg, about 20 to about 70 mg, about 25 to about 70 mg, about 30 to about 70 mg, about 35 to about 70 mg, about 40 to about 70 mg, about 45 to about 70 mg, about 50 to about 70 mg, about 55 to about 70 mg, about 60 to about 70 mg; about 0.1 to about 60 mg, about 1 to about 60 mg, about 5 to about 60 mg, about 10 to about 60 mg, about 15 to about 60 mg, about 20 to about 60 mg, about 25 to about 60 mg, about 30 to about 60 mg, about 35 to about 60 mg, about 40 to about 60 mg, about 45 to about 60 mg, about 50 to about 60 mg; about 0.1 to about 50 mg, about 1 to about 50 mg, about 5 to about 50 mg, about 10 to about 50 mg, about 15 to about 50 mg, about 20 to about 50 mg, about 25 to about 50 mg, about 30 to about 50 mg, about 35 to about 50 mg, about 40 to about 50 mg; about 0.1 to about 40 mg, about 1 to about 40 mg, about 5 to about 40 mg, about 10 to about 40 mg, about 15 to about 40 mg, about 20 to about 40 mg, about 25 to about 40 mg, about 30 to about 40 mg; about 0.1 to about 30 mg, about 1 to about 30 mg, about 5 to about 30 mg, about 10 to about 30 mg, about 15 to about 30 mg, about 20 to about 30 mg; about 0.1 to about 20 mg, about 0.1 to about 10 mg, about 1 to about 20 mg, about 5 to about 20 mg, or about 10 to about 20 mg, including ranges therebetween, per unit dosage in a suitable carrier or excipient.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety for all purposes. Such formulations will contain a therapeutically effective amount of a biologically active agent described herein, which can be in purified form, together with a suitable amount of carrier to provide the form for proper administration to the subject.

The term “formulation” refers to preparing a drug in a form suitable for administration to a subject, such as a human. Thus, a “formulation” can include pharmaceutically acceptable excipients, including diluents or carriers. Pharmaceutically acceptable excipients for use in the compositions of the present disclosure are selected based upon a number of factors including the particular compound used, and its concentration, stability and intended bioavailability; the subject, its age, size and general condition; and the route of administration.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)), which are incorporated herein in its entirety for all purposes. Except insofar as any conventional media or agent is incompatible with an active ingredient, its use in the pharmaceutical compositions is contemplated. Supplementary active ingredients can also be incorporated into the pharmaceutical compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. Routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the route of administration is intrathecal. For example, the agents of use with the current disclosure can be formulated by known methods for administration to a subject using several routes including: parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Suitable excipients include pharmaceutically acceptable excipients, such as diluents, binders, fillers, buffering agents, pH modifying agents, disintegrants, dispersants, preservatives, lubricants, taste-masking agents, flavoring agents, coloring agents, coatings (e.g., an enteric coating) or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isomorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof. In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The pharmaceutical compositions can be formulated, for example, for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. To facilitate oral administration, the pharmaceutical composition may be prepared with an enteric coating to allow passage through an acidic gastric milieu.

The pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated (e.g., using an enteric coating) to delay disintegration and absorption.

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal (intrathecal), intraperitoneal, or intrasternal routes. Dosage forms suitable for parenteral administration include injectable, sterile solutions, preferably oily or aqueous solutions, as well as suspensions, dispersions, emulsions, or implants, including suppositories. Ampules are convenient unit dosages.

The pharmaceutical compositions can also be formulated for intrathecal administration. Dosage forms suitable for intrathecal administration include solutions, suspensions, dispersions, emulsions or any other dosage form that can be administered or injected into the spinal theca.

Compounds described herein can be prepared as a salt. “Salt” as used herein refers to pharmaceutically acceptable salts of the compounds described herein which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977), which is incorporated herein in its entirety for all purposes. Examples of pharmaceutically acceptable salts include, but are not limited to, nontoxic acid addition salts which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphor-sulfonate, citrate, cyclopentanepropionate, digluconate, dodecylsulfate, ethanesulfonate, formate, fumarate, gluco-heptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

In other embodiments, the compounds may be prepared as “prodrugs” in a pharmaceutically acceptable composition/formulation. As used herein, the term “prodrug” refers to a derivative of a compound that can hydrolyze, oxidize, or otherwise react under biological conditions (in vitro or in vivo) to provide a compound as described herein. Prodrugs may only become active upon some reaction under biological conditions, but they may have activity in their unreacted forms. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs and their uses are well known in the art (see, e.g., Berge, et al. 1977 J. Pharm. Sci. 66:1-19). Prodrugs can typically be prepared using well-known methods, such as those described in Burger's Medicinal Chemistry and Drug Discovery (1995, Manfred E. Wolff ed., 5th ed. 172-178, 931-932), which is incorporated herein in its entirety for all purposes.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Methods of Use

The compounds provided herein are useful in the therapeutic treatment of neurological insult such as stroke, traumatic brain injury, epilepsy as well as for pain and neurodegenerative disease (collectively, “neuro-stress”). Particular note is made of the prophylactic treatment of neuro-stress by administering a therapeutic dosage in advance of insult. This method is available, for example, prior to a surgical procedure that would invade the brain. Without being bound by any particular theory it is believed stroke and other neurological insults entail brain cells being starved of oxygen, glucose, nutrients and an associated accumulation of waste materials, including glutamate, leading to an excitotoxic insult. The neurons of the stroke patient become hyper-excitable due to the release of large concentrations of glutamate (10-fold greater than basal), which activates receptors including extrasynaptic NMDA receptors. This leads to the gating of large amounts of calcium though these normally quiet receptors. The intracellular calcium concentration rises from basal levels (0.05-0.2 μM) to a level (1 μM), which is believed to trigger the activation of cell death pathways and the result is brain damage to the affected areas of the brain.

Accordingly, a method is provided for treating conditions of neuro-stress, the method comprising administering a therapeutically effective amount of a compound described herein to a subject in need thereof. The neuro-stress can include, but is not limited to, depression, autism, schizophrenia, stroke, nerve crush, traumatic brain injury, epilepsy, pain or neurodegenerative disease. The neurodegenerative disease can be selected from the group consisting of retinal degeneration, Alzheimer's and ALS.

Particular note is made of drug administration which, at therapeutic levels, interferes with the signal mediated by the rising levels of calcium so that the elevated calcium does not trigger the cell death pathway. As the ischemic condition abates and calcium levels return to normal the patient recovers and brain damage has been averted.

Compounds of Formula (I) are believed to be usefully administered at therapeutic doses within about 30 minutes to 1 hour of insult and/or neuro-stress. In some instances, an oral formulation, a nasal spray or an acute injection given i.v. or intrathecally to induce a therapeutic concentration in the brain is indicated. Desirably, the pharmaceutical compositions, regardless of the form, protect neural tissue.

It will be understood a therapeutically effective dose will vary according to indication, dosing frequency or schedule, and route of administration, according to a consideration of factors as well known in the art.

The therapeutically effective dose can generally be from 0.1 to 100 mg/kg/day, from 0.1 to 90 mg/kg/day, from 0.1 to 80 mg/kg/day, from 0.1 to 70 mg/kg/day, from 0.1 to 70 mg/kg/day, from 0.1 to 60 mg/kg/day, from 0.1 to 50 mg/kg/day, from 0.1 to 40 mg/kg/day, from 0.1 to 30 mg/kg/day, from 0.1 to 20 mg/kg/day or from 0.1 to 10 mg/kg/day; from 1 to 100 mg/kg/day, from 1 to 90 mg/kg/day, from 1 to 80 mg/kg/day, from 1 to 70 mg/kg/day, from 1 to 70 mg/kg/day, from 1 to 60 mg/kg/day, from 1 to 50 mg/kg/day, from 1 to 40 mg/kg/day, from 1 to 30 mg/kg/day, from 1 to 20 mg/kg/day or from 1 to 10 mg/kg/day. For example, the therapeutically effective dose can be 0.1 to 10 mg/kg/day. By way of non-limiting example, the therapeutically effective dose for treating cardiac insufficiency or hypertension can be 25-50 mg/kg TID or 0.1-10 mg/kg TID, BID, daily, weekly, or monthly via oral administration. Alternatively, the therapeutically effective dose for treating cardiac insufficiency or hypertension can be 0.1-10 mg, 1-10 mg, 1-6 mg, 5-10 mg, 1-100 mg, 1-500 mg, 1-1000 mg, 1-1500 mg, 1-1600 mg, 1-2000 mg, administered daily via IV bolus and/or IV infusion.

### Methods of Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration. For example, administration can be intrathecal, parenteral, oral, buccal, sublingual or nasal. Preferably, administration is intrathecal.

Compounds of Formula (I) and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydro-gels, liposomes, micelles (e.g., up to 30 mm), nanospheres (e.g., less than 1 nm), microspheres (e.g., 1-100 μm), reservoir devices, a combination of any of the above, or other suitable delivery vehicles to provide the desired release profile in varying proportions. Other methods of controlled-release delivery of compounds of Formula (I) or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Compounds of Formula (I) can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331), which is incorporated herein in its entirety for all purposes. Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with agents or excipients; improve the stability of the compounds of Formula (I) in vivo; prolong the residence time of the compounds of Formula (I) at its site of action by reducing clearance; decrease the nonspecific delivery of the compounds of Formula (I) to nontarget tissues; decrease irritation caused by the compounds of Formula (I); decrease toxicity due to high initial doses of the compounds of Formula (I); alter the immunogenicity of the compounds of Formula (I); decrease dosage frequency, improve taste of the product; or improve shelf life of the product.

Pharmaceutical compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253), which are each incorporated herein in their entirety for all purposes.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended.

For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such reference is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples

### REFERENCES

1. Li et al., “Thermodynamic profiling of conformationally constrained cyclic ligands for the PDZ domain,” Bioorganic & Medicinal Chemistry Letters, 14, 1385-1388 (2004)
2. Guy et al. “Small molecule inhibition of PDZ-Domain interaction,” U.S. Pat. No. 7,141,600.
3. Chamila N. Rupasinghe and Mark R. Spaller, “The interplay between structure-based design and combinatorial chemistry,” Current Opinion in Chemical Biology, 10, 188-193 (2006).
4. Sharma et al., “Design, synthesis, and evaluation of linear and cyclic peptide ligands for PDZ10 of the multi-PDZ domain protein MUPP1,” Biochemistry, 46, 12709-12720 (2007).
5. Cui et al. “PDZ protein interactions underlying NMDA receptor mediated excitotoxicity and neuroprotection by PSD-95 inhibitors,” J. Neuroscience, 27, 9901-9915 (2007).
6. Klosi et al., “Bivalent peptides as PDZ domain ligands,” Bioorganic & Medicinal Chemistry Letters, 17, 6147-6150 (2007).
7. Saro et al. “A thermodynamic ligand binding study of the third PDZ domain (PDZ3) from the Mammalian Neuronal Protein PSD-95,” NIH, 46, 6340-6352. (2008).
8. Gomika et al. “A chemical library approach to organic-modified peptide ligands for PDZ domain proteins: A synthetic, thermodynamic and structural investigation,” Chem. Bio. Chem., 9, 1587-1589 (2008).
9. D. Gomika Udugamasooriya and Mark R. Spaller “Conformational constraint in protein ligand design and the inconsistency of binding entropy.” Biopolymers, 89, 653-667 (2008).
10. Tao Li, “Studies of ligand-protein interaction for the third PDZ domain (PDZ3) of mammalian post-synaptic density-95 (PSD-95)” (Jan. 1, 2005). ETD Collection for Wayne State University. Paper AAI3198697. http://digitalcommons.wayne.edu/dissertations/AAI3198697
11. Sutcliffe-Goulden et al., “Receptor-binding cyclic peptides and methods of use,” U.S. Ser. No. 11/198,884 (2005).
12. Darwish et al., “Efficient synthesis of CN2097 using in situ activation of sulfhydryl group” Tetrahedron Lett. 2017 Aug. 2; 58(31): 3053-3056.

The above references are incorporated herein in their entirety for all purposes.

### EXAMPLES

The following non-limiting examples are provided to further illustrate the present disclosure.

### Example 1: Synthesis of Peptides

### Example 1a: Synthesis of (N-Dde)-c[-Lys-Thr-Glu((βAla)-]-Val

Step I. Val-NovaSyn TGA resin (n mmol) was loaded into a peptide synthesis reaction vessel. The resin was suspended and swelled (45 min) with shaking using dichloromethane (DCM).
Step II. Deprotection of the Fmoc group was carried out by adding piperidine-DMF (20% v/v; 10× resin volume) for 10 minutes. Then, piperidine-DMF was drawn off and the resin was treated with fresh piperidine-DMF (20% v/v; 10× resin volume) for another 10 min while shaking. The solvent was drawn off, and the resin was washed with DMF (10× resin volume).
Step III. Amino acid precursor (3 equiv), DIPCI (4 equiv), HOBt (6 equiv) and DMF (5× resin volume) were mixed within the reaction vessel and shaken gently. After 2 h, a small amount of resin sample was removed, placed in a test tube and washed sequentially with DMF (3×1 mL), DCM (3×1 mL) and ether (2×1 mL). The washed sample was submitted to the Kaiser Test. A negative result was indicative of complete coupling.
Step IV. If the reaction is not complete, additional DIPCI (1 equiv) was added and the vessel shaken for another 2 h. If the coupling was complete, the solution was drawn off and the resin was washed with DMF (10× resin volume).

Steps I-IV These protocols were used to synthesize the peptides KTEV (SEQ ID NO: 12) using Dde-Lys(Fmoc)OH and Fmoc-Glu(PhiPr)OH for the P-3 and P-1 positions, respectively (Scheme 1). The cyclization was accomplished as follows. Using step II, the lysine side chain Fmoc group was removed. Using step III in the linear peptide synthesis protocol, Fmoc-β-alanine (linker) was coupled to the side chain amino group of Lys. The PhiPr protecting group from glutamate was removed using cleavage solution (5× resin volume, TFA/EDT/thioanisole/anisole/DCM (2:4:1:1:92 v/v)) for 30 min. The solution was drawn off and the resin was treated with fresh cleavage solution for another 30 min. This procedure was repeated another two times and finally washed with DMF (10×10× resin volume). A malachite green test was carried out to confirm the presence of free COOH in the resin. Green beads are indicative of free COOH groups. Using step II, the Fmoc group on (3-alanine was removed. An aggregation of beads was seen since there is a possibility of forming salt bridges or ionic interactions between the free amino groups and carboxylic acid groups. Then, the resin was washed three times with DIPEA in DMC (10× resin volume, 1:9 v/v; 3 min each time) to wash away any trace piperidine. Finally, the resin was washed with DMF (10× resin volume). This step was followed by the addition of solvent mixture DMSO-N-methylpyrrolidone (NMP) (10× resin volume, 1:4 v/v), which was added to the reaction vessel and gently shaken until the aggregated resin spread out and was evenly suspended in solvent. HBTU (3 equiv), DIEPA (6 equiv), and HOBT (3 equiv) were then added and the vessel shaken gently (Scheme 1).

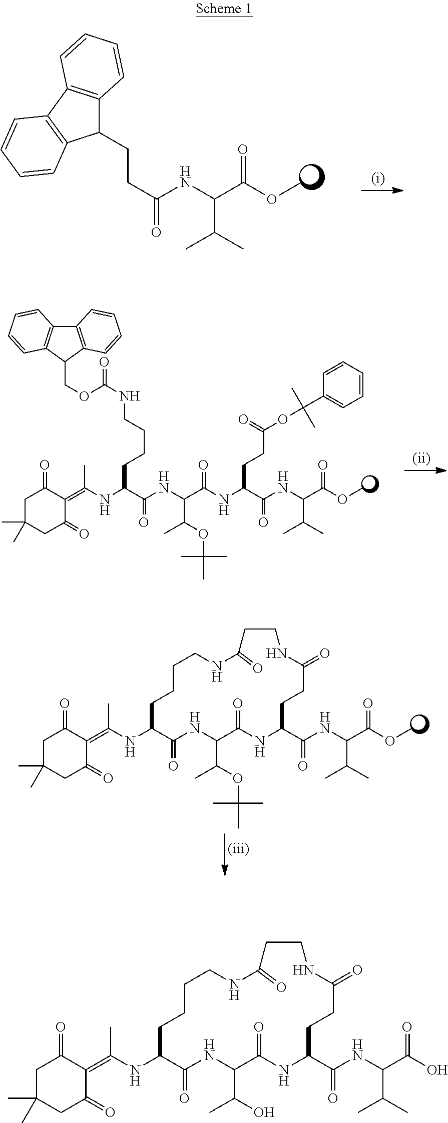

After 2 h, a small amount of resin sample (4-5 mg) was removed and placed in a small test tube, and Kaiser's Test was carried out to check the reaction progress. A negative Kaiser's test was indicative of complete cyclization. If the reaction was complete, the resin was washed with DMF (10×10× resin volume). If the reaction was not complete, the solvent was drawn off and fresh DMSO-N-methylpyrrolidone (NMP) (10× resin volume, 1:4 v/v) was added with HBTU (3 equiv), DIEPA (6 equiv), and HOBT (3 equiv) and the cyclization was repeated (Scheme 2). Whether the reaction was finished or not in the allocated time, the resin was washed with DMF (10×1 mL) in order to avoid epimerization. Finally the resin was washed with DCM (5×10× resin volume). Then, a small amount of resin sample (^{˜}20 mg) was removed and resin cleavage solution (5× resin volume, TFA/TIS (triisopropylsilane)/thioanisole/anisole (92:4:2:2, v/v)) was added with shaking for 2 h to obtain compound A (Scheme 1). The characterization of compound A (below) was (N-Dde)-c[-Lys-Thr-Glu(βAla)-]-Val C33H52N6O10; Predicted [M+H]+: 694.0; ESI-MS found: [M+H]+ 693.0, [M+Na]+715.0. Compound A had the structure shown below.

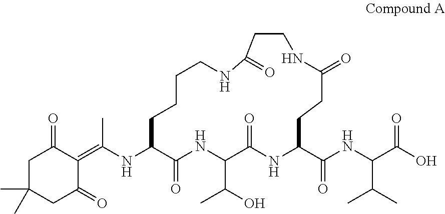 [SMILES: CC(N[C@H]1CCCCNC(=O)CCNC(=O)CC[C@@H](C(=O)NC(C(=O)O)C(C)C)NC(=O)C(C(C)O)NC1=O)=C1C(=O)CC(C)(C)CC1=O]

### Example 1b: Synthesis of a Non-Limiting Y Group

Synthesis of Cys-Gly-Ser-Phe-Pro-Pro-Trp-Val-Gln-c[-Lys-Thr-Glu (β-Ala)-]-Val. When the cyclization was completed as described for the synthesis of Compound A, the extension of the peptide was carried out by first removing the Dde protecting group from the N-terminus lysine. This deprotection was affected by a deprotection solution (10× resin volume, hydrazine/DMF (3% v/v), 10 min). After 10 min solution was drawn off with a weak vacuum and fresh deprotection solution (10× resin volume, hydrazine/DMF (3% v/v), 10 min) was added while shaking. The Dde group removal procedure was repeated two more times, followed by washing with DMF (10×10× resin volume). The Kaiser's test was performed to confirm Dde removal. A positive result was indicative of the free amino group. Steps II and III were repeated with amino acid residues (Fmoc protected if necessary) until the desired peptide was synthesized. Step II was used to remove the Fmoc group from the N-terminus. Then, a small amount of resin sample (−20 mg) was removed before and after Fmoc-Cys (Trt)-OH coupling and resin cleavage solution (5× resin volume, TFA/TIS (triisopropylsilane)/thioanisole/anisole (92:4:2:2, v/v)) was added with shaking for 2 h to obtain Compound B.

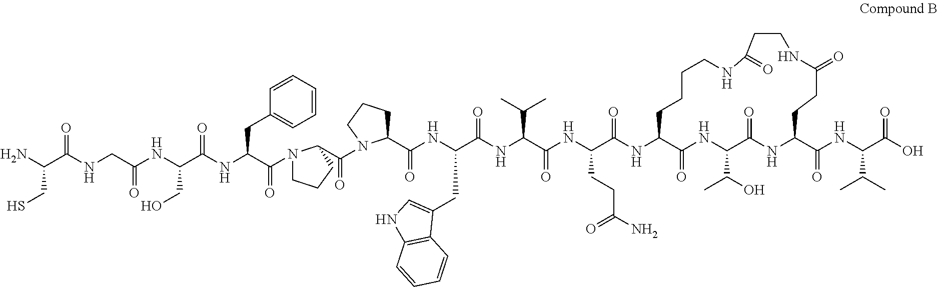 [SMILES: CC(C)[C@H](NC(=O)[C@@H]1CCC(=O)NCCC(=O)NCCCC[C@H](NC(=O)[C@H](CCC(N)=O)NC(=O)[C@@H](NC(=O)[C@H](Cc2c[nH]c3ccccc23)NC(=O)[C@@H]2CCCN2C(=O)[C@H]2CCCN2C(=O)[C@H](Cc2ccccc2)NC(=O)[C@H](CO)NC(=O)CNC(=O)[C@@H](N)CS)C(C)C)C(=O)N[C@@H](C(C)O)C(=O)N1)C(=O)O]

### Example 1c: Synthesis of a Non-Limiting z Group

The procedure for this peptide was similar to the general linear peptide syntheses, which was manually prepared and purified using standard Fmoc-based solid-phase peptide synthesis protocols. The synthesis of oligoarginine was a particularly difficult synthetic procedure compared to standard linear peptide synthesis. The synthesis was carried out on Rink Amide AM resin (n mmol). The coupling steps were more difficult, and following the coupling of several arginine residues, the beads aggregate after Fmoc-deprotection. The coupling protocol was modified to solve the problem by using pre-activated solutions of arginine.

Specifically, to couple arginine, pre-activation was accomplished by combining DIPEA (4 equiv), HOBT (2.5 equiv), HBTU (2.5 equiv) and DMF (5× resin volume), which were shaken gently for 7 min. Arginine precursor (3 equiv) was then added. After 2 h, a small amount of resin sample (4-5 mg) was removed and submitted to the Kaiser Test. A negative result was indicative of complete coupling. After the coupling was complete, the reaction solution was drawn off and resin was washed with DMF (10×10× resin volume). Fmoc group removal was effected by the standard procedure, followed by washing with DCM to remove trace DMF (10×10× resin volume). Coupling of arginine was carried out another six times. Small amount of resin beads were cleaved off to confirm the synthesis of compound C. The cleavage was carried out similar to standard protocols using resin cleavage solution (5× resin volume, TFA/TIS (triisopropylsilane)/anisole (92:6:2, v/v)) after shaking for 28 h. The characterization for compound C was (N-Fmoc)-Arg-Arg-Arg-Arg-Arg-Arg-Arg-CONH2; C57H97N29O9; Predicted [M+H]+: 1333.5; MALDI-TOF-MS found: [M+H]+ 1332.8; [M-156]+1176.5, [M-312]+1020.5. The structure for compound C is shown below.

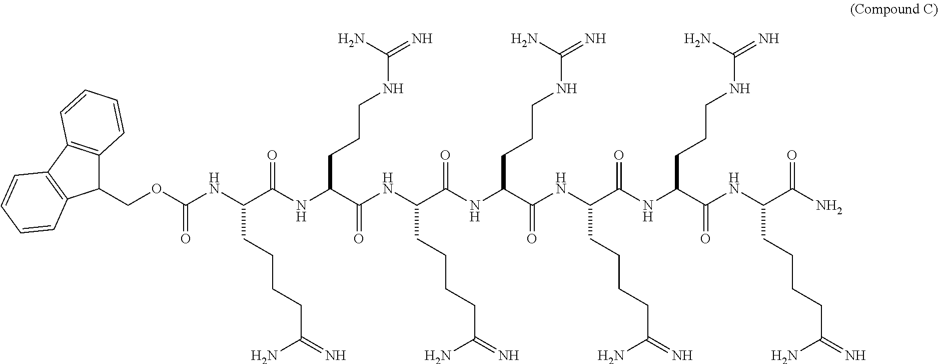 [SMILES: N=C(N)CCCC[C@H](NC(=O)[C@H](CCCNC(=N)N)NC(=O)[C@H](CCCCC(=N)N)NC(=O)[C@H](CCCNC(=N)N)NC(=O)[C@H](CCCCC(=N)N)NC(=O)[C@H](CCCNC(=N)N)NC(=O)[C@H](CCCCC(=N)N)NC(=O)OCC1c2ccccc2-c2ccccc21)C(N)=O]

### Example 1d: Synthesis of Another Non-Limiting z Group

This procedure was the same as the synthesis for Example 1c, but included an additional coupling of Boc-Cys(Npys)-OH or Fmoc-Cys(Npys)-OH to the N-terminus using general coupling protocols. The characterization for compound D was Cys(Npys)-Arg-Arg-Arg-Arg-Arg-Arg-Arg-CONH2; C50H94N32010S2; Predicted [M+H]+: 1367.5; [M+H-(Npys)]+1213.5; ESI-MS found: [M+H-(Npys)]+1213.5; [M+H-(Npys)-156]+1057.5; [M+H-(Npys)-312]+901.0. The structure for compound D is shown below.

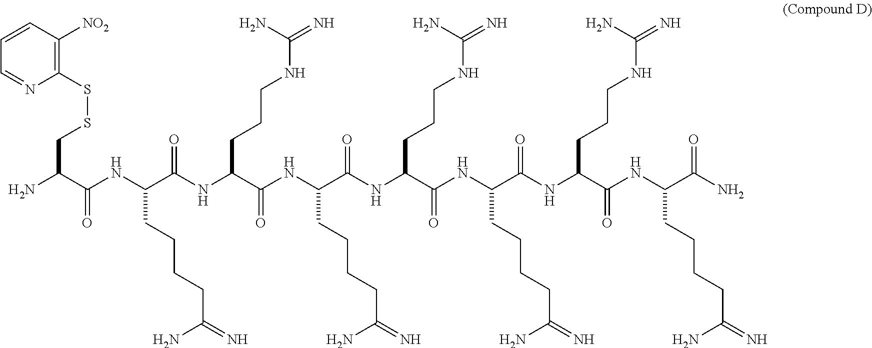 [SMILES: N=C(N)CCCC[C@H](NC(=O)[C@H](CCCNC(=N)N)NC(=O)[C@H](CCCCC(=N)N)NC(=O)[C@H](CCCNC(=N)N)NC(=O)[C@H](CCCCC(=N)N)NC(=O)[C@H](CCCNC(=N)N)NC(=O)[C@H](CCCCC(=N)N)NC(=O)[C@@H](N)CSSc1ncccc1[N+](=O)[O-])C(N)=O]

### Example 2: Compounds of Formula (IV) are Superior to CN2097 in Enhancing TrkB Signaling

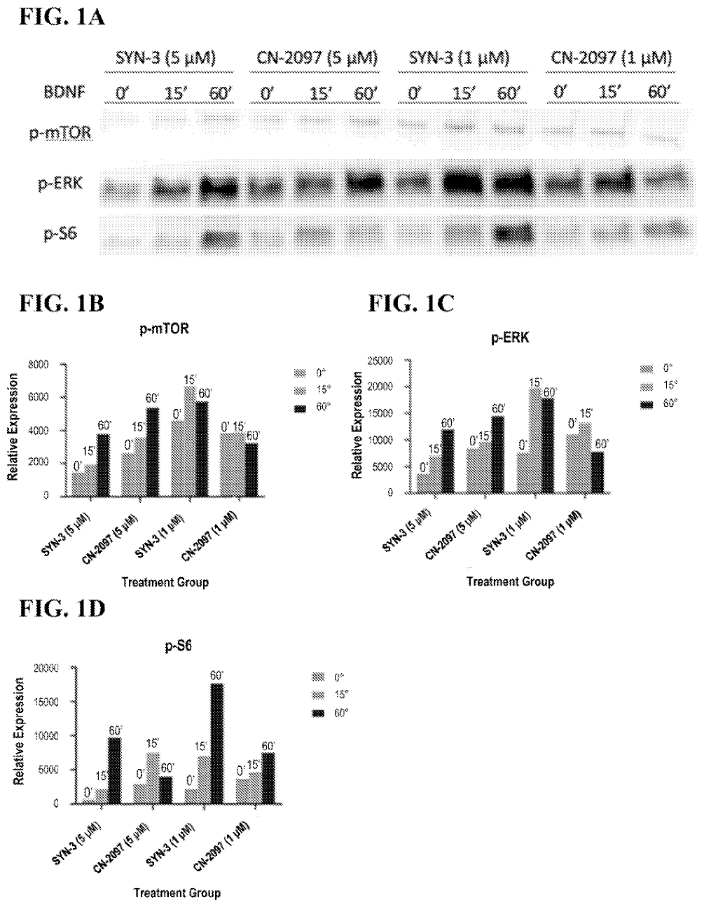

SH-SY5Y cells were transfected with HA-TrkB. Following transfection, cells were starved in MEM for 3 hours. In the last 90 minutes of starvation, cells were treated with compounds of Formula (IV) or CN-2097 (shown below) at 1 μM and 5 μM. In the last hour of starvation, cells were stimulated with BDNF. Western blots probed with phospho-specific antibodies against p-MTOR, p-Erk and p-S6 (FIG. 1A).

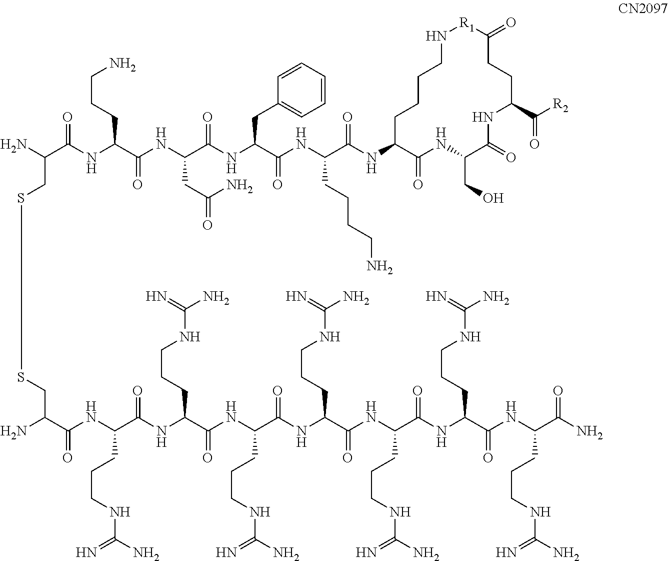

Compounds of Formula (IV) had a similar effect as CN2097 on phosphorylated MTOR and phosphorylated Erk (FIG. 1B and FIG. 1C). However, a 5-fold lower dose of compounds of Formula (IV) (1 μM) compared to CN2097 enhanced p-S6 activity (FIG. 1D).

### Example 3: Compounds of Formula (IV) Restores Long-Lasting Increases in Synaptic Strength (LTP)

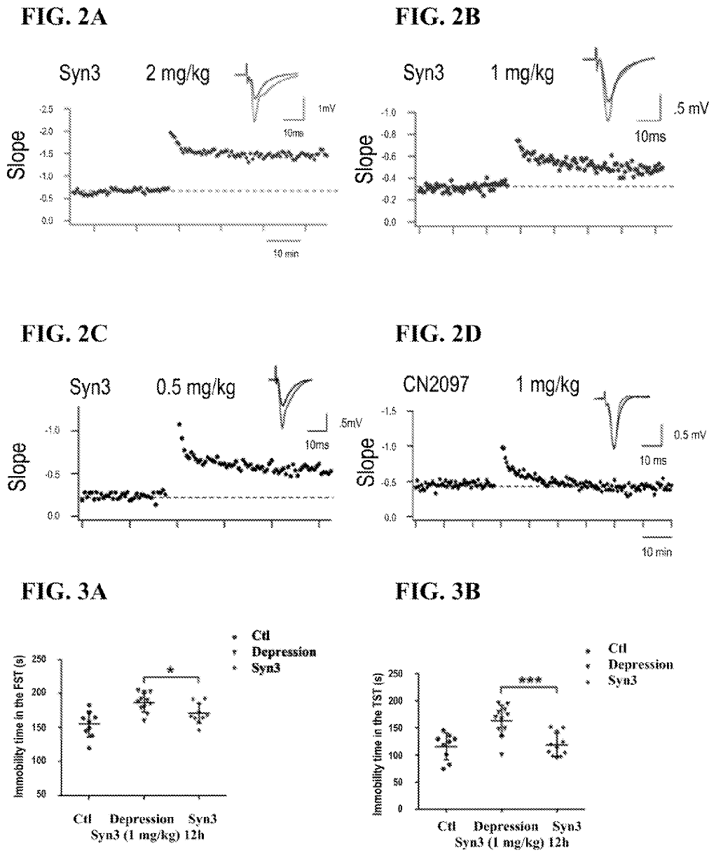

As shown in FIG. 2A-2C, compounds of Formula (IV) restored hippocampal LTP in an Angelman mouse model at a significantly lower dose as compared to CN2097. Field EPSP slopes were recorded in the CA1 region of the hippocampus.

Electrophysiological recordings and data analysis were performed as described in Cao et al., 2013 (Impairment of TrkB-PSD-95 Signaling in Angelman Syndrome. PLoS Biol 11, e1001478 (2013). Coronal brain slices (350 μm) were maintained in an interface chamber containing oxygenated artificial cerebral spinal fluid (ACSF) for at least 1 h before use. Slices were transferred to a submersion recording chamber perfused with oxygenated ACSF at 2 ml/min. Extracellular postsynaptic field potentials (fEPSPs) were performed by placing electrodes (borosilicate glass, resistance <1 MΩ) in the CA1 stratum radiatum and synaptic responses elicited by stimulation of the Schaffer Collaterals using concentric bipolar stimulation electrodes and recorded using an AxoClamp2B amplifier (Axon instruments) and EX1 differential amplifier (Dagan), and digitized at 10 kHz. Data were acquired using Igor Pro (Wave Metrics) and Neuromatic (neuromatic.thinkrandom.com). LTP was induced by high frequency stimulation (2×HFS for is at 100 Hz separated by 20 s) of Schaffer collateral afferents and LTP values are expressed as percentage of baseline±SEM. Paired two-tailed t-tests were used for statistical analysis.

No LTP was observed in CN2097 intraperitoneal (IP) injected AS mice at 1 mg/kg (lower trace) (black dots) (FIG. 2D). IP injection of compounds of Formula (IV) into AS mutant mice at 0.5 mg/kg resulted in LTP (FIG. 2C).

### Example 4: The Effects of i.v. Compounds of Formula (IV) (Syn3) on the Depressive-Like Behavior of Repeated Corticosterone-Injected Mice

Preclinical identification of fast-onset antidepressants requires animal models that can accurately predict the delay to therapeutic onset. Significantly, depression models have proven ability to differentiate the effects of rapidly acting anti-depressants, such as ketamine, as opposed to SSRIs. This has led to the approval of SPRAVATO® (esketamine), a ketamine-derived drug which is the first antidepressant with a novel mechanism of action approved in decades. The neurobiological changes believed to underlie depressive symptomatology have been reliably characterized using exogenous corticosterone (CORT) administration e.g. dendritic atrophy in hippocampal pyramidal cells with loss of CA3 and CA1 apical dendrites. As shown in FIG. 3, we examined the effects of i.v. compounds of Formula (IV) on the depressive-like behavior of repeated corticosterone-injected mice. Mice then received a single injection of compounds of Formula (IV) (1 mg/kg) or Vehicle (0.9% NaCl). Day 7 after injection of compounds of Formula (IV), immobility times in the FST and TST were tested as described. #P<0.001 vs. control mice. As shown in FIG. 3A and FIG. 3B, acute injection of compounds of Formula (IV) (e.g., Syn3) reduced the duration of immobility time in both the Forced Swim Test (FIG. 3A) and the Tail Suspension Test (FIG. 3B).

### Example 5: Role of Inflammatory Cytokines in Depression

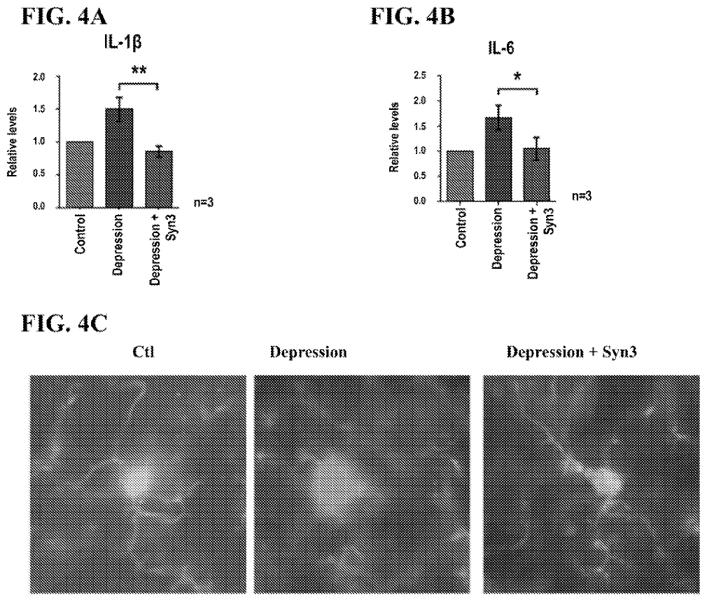

A major breakthrough in our understanding of the mechanistic basis for depression is the “inflammatory hypothesis”. Patients with MDD exhibit increased peripheral blood concentrations of inflammatory cytokines such as interleukin (IL)-1l and IL-6. Mice subjected to chronic corticosterone (CORT) treatment for 4 weeks exhibited depressive-like symptoms, including decreased sucrose preference, concomitantly with increased IL-1β and IL-6 levels (FIG. 4A and FIG. 4B). Notably, the administration of compounds of Formula (IV) (Syn3) blocks the increased expression of IL-1β in the CORT behavioral model (FIG. 4A).

Microglial cells are capable of influencing complex moods, synaptic plasticity, neurogenesis, and memory. Microglial activation has also been demonstrated in depressed patients. Classically activated, pro-inflammatory microglia phenotypes (termed M1-like), often release inflammatory mediators that result in severely injured tissue. Alternatively activated, anti-inflammatory microglial phenotypes (termed M2-like) are neuroprotective in function and promote the survival of new neurons. In a healthy brain, most microglial cells are in a resting state. The morphology of resting microglia is poly-branched with many fine branches and a smaller cytoplasm, compared to active microglia that display an increased soma size and more irregular shape of their cell body. Activation of microglia results in the release of pro-inflammatory factors, namely IL-1β, TNFα and IL-6. As shown in FIG. 4C, administration of compounds of Formula (IV) (Syn3) blocks switches microglia from an active form (CORT depression) to the resting phenotype.

### Example 6: Proposed Treatment of Patient with Ischemia

A 48 year old female presents with an ischemic episode resulting sudden numbness the face, and left arm, confusion, and trouble speaking. Within 2 hours of onset, compositions containing compounds of Formula (IV) will be administered at 50 mg/Kg i.v. Within 48 hours the patient recovers without significant brain damage. Doses of from about 1 to about 100 mg/Kg will be used.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A compound having the structure of Formula (I), a salt or isomer thereof: 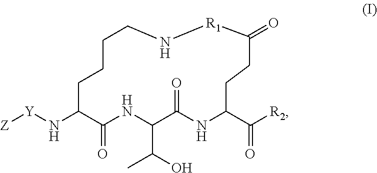
wherein R₁ comprises at least about one amino acid; R₂ is selected from the group consisting of isoleucine, leucine, alanine, phenylalanine, and valine; Y is a peptide chain comprising 1 to 10 amino acids; Z comprises hydrogen or a molecular transporter; and wherein the compound acts as a reversible agonist of the PDZ3 domain of PSD-95.

2. The compound of claim 1, wherein R₁ is alanine or β-alanine.

3. (canceled)

4. The compound of claim 1, wherein R₂ is valine or alanine.

5. (canceled)

6. The compound of claim 1, wherein Y comprises a peptide chain having a length of 3 to 9 amino acids.

7. The compound of claim 1, wherein Y comprises any one of SEQ ID NOs: 1, 2, or 12-17.

8. (canceled)

9. The compound of claim 1, wherein Y does not consist of SEQ ID NO: 3 (CKNYK peptide).

10. The compound of claim 1, wherein Z comprises the molecular transporter.

11. The compound of claim 10, wherein Z comprises a liposome, a steroid, a polyamine, a nanotube, a nanoparticle, a dendrimer, a cell penetrating peptide, a protein-transduction domain amino acid, a peptoid, (N-substituted glycine), an oligocarbamate, an arginine oligomer of about 6 to 20 units, a D-arginine oligomer, a spaced arginine oligomer, a N-arginine peptoid, an oligocarbamate transporter, or a tetrameric dendrimer.

12. The compound of claim 10, wherein Z comprises a D-arginine oligomer, a spaced arginine oligomer, a N-arginine peptoid, an oligocarbamate transporter, a tetrameric dendrimer, a releasable-luciferin-transporter conjugate, a arginine oligomer of about 6 to 20 units, or a cell penetrating peptide.

13-15. (canceled)

16. The compound of claim 11, wherein Z comprises the cell penetrating peptide and the cell penetrating peptide comprises any one of SEQ ID NOs: 4 to 11.

17. The compound of claim 1, wherein Y and Z each comprise a cysteine and wherein Y and Z are connected by a disulfide bond.

18. The compound of claim 17, wherein Y and Z together form Structure (Ia) or Structure (Ib):

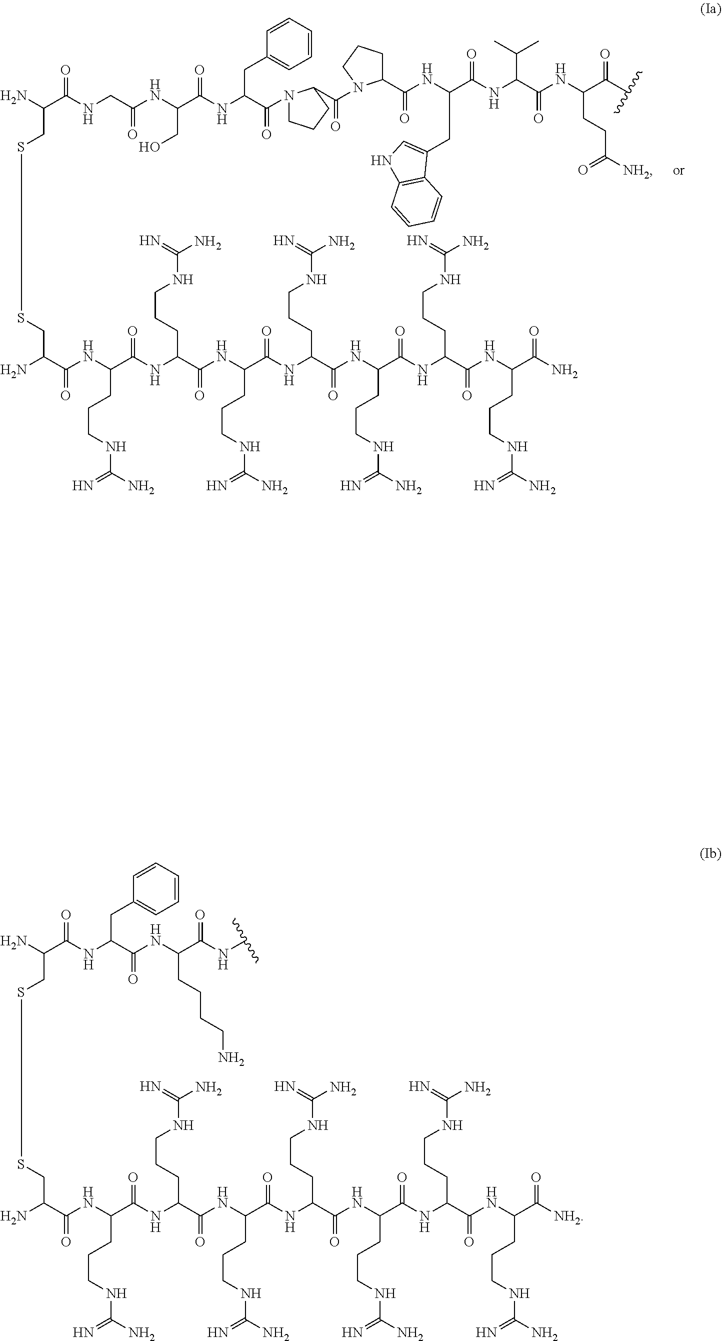 [SMILES: *C(=O)C(CCC(N)=O)NC(=O)C(NC(=O)C(Cc1c[nH]c2ccccc12)NC(=O)C1CCCN1C(=O)C1CCCN1C(=O)C(Cc1ccccc1)NC(=O)C(CO)NC(=O)CNC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O)C(C)C ; *NC(=O)C(CCCCN)NC(=O)C(Cc1ccccc1)NC(=O)C(N)CSSCC(N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O]

19. The compound of claim 1, wherein the compound has a structure in accordance with compounds of Formula (IV) or Formula (V):

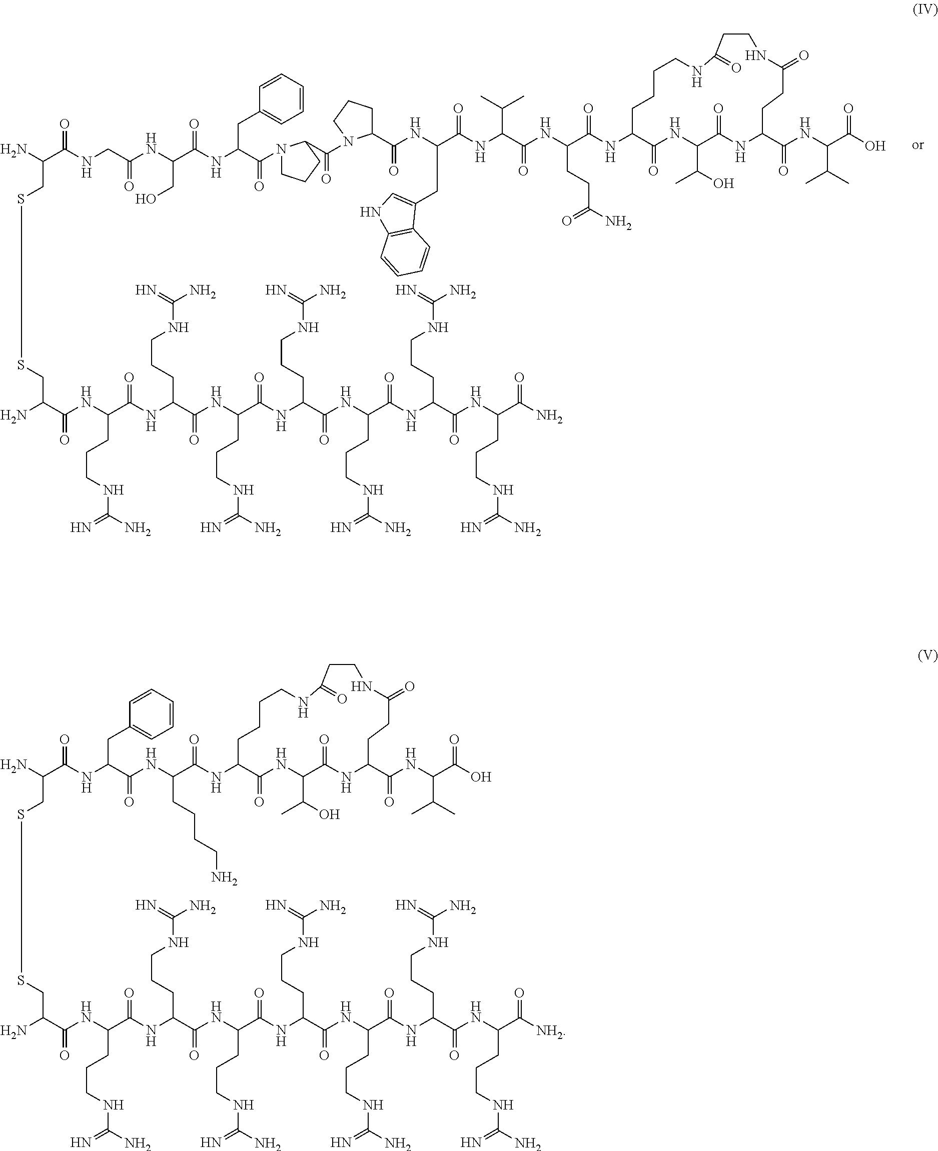

20. The compound of claim 1, wherein the compound is selected from:

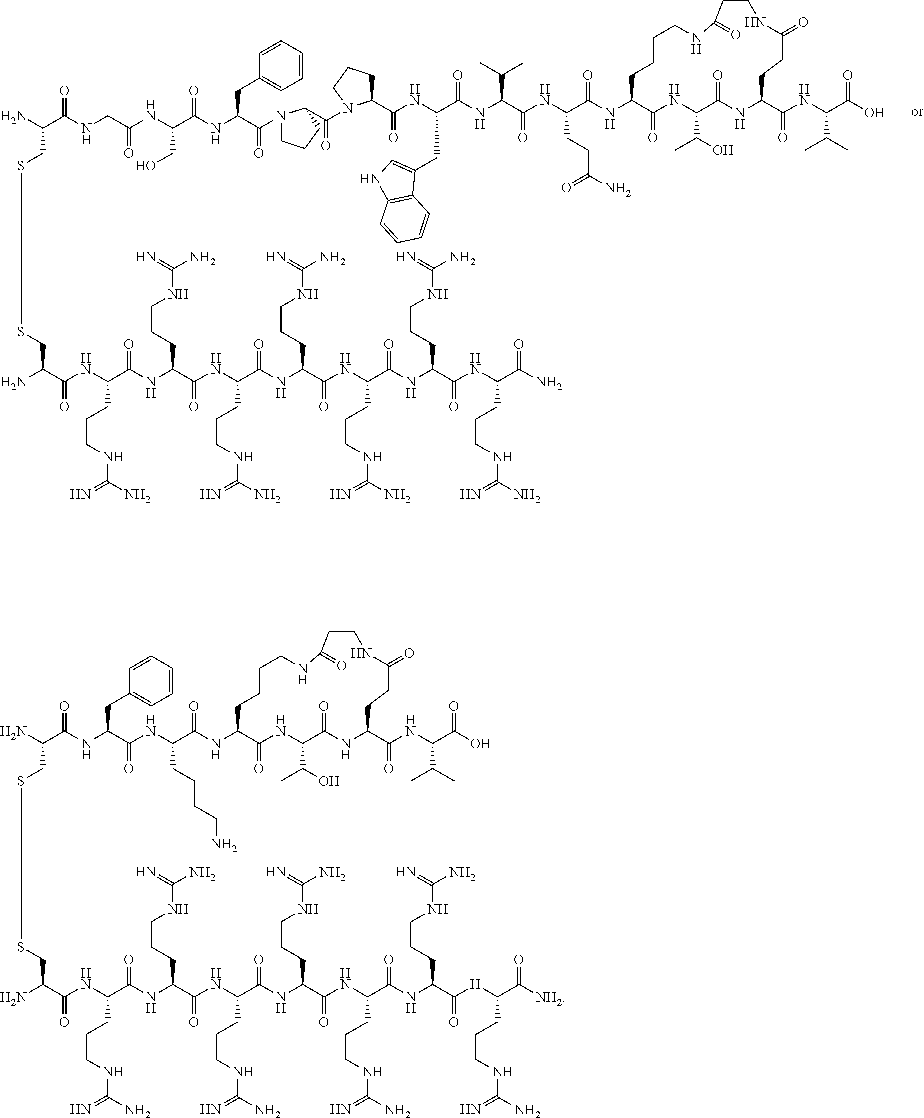

21. A reversible agonist composition for the PDZ3 domain of PSD-95 comprising the compound of claim 1 and a pharmaceutically acceptable excipient.

22. The composition of claim 21, wherein the composition is prepared as a unit dosage form comprising from about 0.1 to about 100 mg of the compound of any one of claims 1 to 18 in the pharmaceutically acceptable excipient per unit dosage.

23. A method of treating neuro-stress in a subject in need thereof, the method comprising: administering to the subject an effective amount of the compound of claim 1.

24. The method of claim 23, wherein the neuro-stress is selected from the group comprising depression, autism, schizophrenia, stroke, nerve crush, traumatic brain injury, epilepsy, pain or neurodegenerative disease.

25. The method of claim 24, wherein the neurodegenerative disease is selected from the group consisting of retinal degeneration, Alzheimer's, and ALS.

26. The method of claim 23, wherein the effective amount of the compound is from about 0.1 mg/kg to about 100 mg/kg according to the mass of the subject, wherein the route of administration is selected from intrathecal, parenteral, oral, buccal, sublingual and nasal.

27-28. (canceled)


SEQUENCE LISTING (17 sequences)

SEQ ID NO: 1 - protein, 9 aa, Artificial Sequence
  synthetic construct
CGSFPPWVQ

SEQ ID NO: 2 - protein, 3 aa, Artificial Sequence
  synthetic construct
CYS

SEQ ID NO: 3 - protein, 5 aa, Artificial Sequence
  synthetic construct
CKNYK

SEQ ID NO: 4 - protein, 9 aa, Artificial Sequence
  synthetic construct - cell penetrating peptide
RRRRRRRRR

SEQ ID NO: 5 - protein, 9 aa, Artificial Sequence
  synthetic construct - cell penetrating peptide
RKKRRQRRR

SEQ ID NO: 6 - protein, 16 aa, Artificial Sequence
  synthetic construct - cell penetrating peptide
RQIKIWFQNRRMKWKK

SEQ ID NO: 7 - protein, 21 aa, Artificial Sequence
  synthetic construct - cell penetrating peptide
KETWWETWWTEWSQPKKKRKV

SEQ ID NO: 8 - protein, 27 aa, Artificial Sequence
  synthetic construct - cell penetrating peptide
GWTLNSAGYLLGKINLKALAALAKKIL

SEQ ID NO: 9 - protein, 10 aa, Artificial Sequence
  synthetic construct- nuclear localization sequence
VQRKRQKLMP

SEQ ID NO: 10 - protein, 8 aa, Artificial Sequence
  synthetic construct - nuclear localization sequence
SKKKKIKV

SEQ ID NO: 11 - protein, 8 aa, Artificial Sequence
  synthetic construct - nucelar localization sequence
GRKRKKRT

SEQ ID NO: 12 - protein, 9 aa, Artificial Sequence
  synthetic construct
CGSFPPWVK

SEQ ID NO: 13 - protein, 10 aa, Artificial Sequence
  synthetic construct
CGSFPPWVKK

SEQ ID NO: 14 - protein, 10 aa, Artificial Sequence
  synthetic construct
CGSFPPWVKQ

SEQ ID NO: 15 - protein, 10 aa, Artificial Sequence
  synthetic construct
CGSFPPWVQK

SEQ ID NO: 16 - protein, 10 aa, Artificial Sequence
  synthetic construct
CGSFPPWVQQ

SEQ ID NO: 17 - protein, 9 aa, Artificial Sequence
  synthetic construct
  MISC_FEATURE (1)..(1): X is dCys
  MISC_FEATURE (5)..(5): X is dPro
XGSFXPWVQ